(19) United States
(12) Patent Application Publication
(10) Pub. No.: US 2018/0228880 A1
(43) Pub. Date: Aug. 16, 2018

(54) Materials and Methods for Eliciting Targeted Antibody Responses In Vivo

(71) Applicant: THE REGENTS OF THE UNIVERSITY OF MICHIGAN, Ann Arbor, MI (US)

(72) Inventors: Stephen J. Weiss, Ann Arbor, MI (US); David T. Dudley, Ann Arbor, MI (US)

(21) Appl. No.: 15/712,945

(22) Filed: Sep. 22, 2017

Related U.S. Application Data
(62) Division of application No. 14/642,246, filed on Mar. 9, 2015, now abandoned.
Application No. 14/642,246 is a continuation-in-part of application No. 13/474,872, filed on May 18, 2012, now Pat. No. 8,975,029.
(60) Provisional application No. 61/487,812, filed on May 19, 2011.

(51) Int. Cl. A61K 39/00; G01N 33/68; C07K 16/30; A61K 35/36; C07K 16/28

(52) U.S. Cl. CPC A61K 39/0011; C07K 2317/34; G01N 33/6854; A61K 2039/505; C07K 16/3015; C07K 16/2839; G01N 2333/7055; A61K 35/36; C07K 2317/73; C07K 2317/76; C07K 16/30

(57) ABSTRACT

Methods for generating and identifying antibodies specifically binding target molecules expressed by cells embedded in a three-dimensional extracellular matrix resembling the in vivo environment and form of the target are provided. Also provided are methods of producing immunogens that yield targets in such forms. Further provided are methods for identifying anti-cancer therapeutics, such as antibody products. Hydrogels are also provided, and those hydrogels may comprise a cross-linked protein are also provided. Diagnostics, prophylactics and therapeutics identified using the methods disclosed herein are also provided.

### CROSS-REFERENCE TO RELATED APPLICATIONS

This Application is a Division of application Ser. No. 14/642,246 filed on Mar. 9, 2015. Application Ser. No. 14/642,246 is a Continuation-in-part of application Ser. No. 13/474,872 filed on May 18, 2012. Application Ser. No. 13/474,872 claims the benefit of Provisional U.S. Application 61/487,812 filed on May 19, 2011. The entire contents of these applications are incorporated herein by reference in their entireties.

### FIELD

The disclosure relates generally to medical products and medical procedures, and more specifically to materials and methods for preventing, treating or ameliorating a symptom of a condition characterized by a cell-surface marker, or target, such as cancer.

### BACKGROUND

A deadly characteristic of cancer cells is their ability to proliferate at uncontrolled rates, invade local tissues, and metastasize to distant sites where they grow anew. Presently, there are few cancer therapies that effectively target cancer cell growth, invasion or metastasis, either on the market or in development. Clearly, the importance of inhibiting cancer cell proliferation and invasion—at either primary or metastatic sites—is compelling. Attempts to identify new targets for therapeutic intervention or to develop the appropriate drugs have been hampered by the inability of in vitro model systems to accurately recapitulate cancer cell proliferation and/or invasion programs as they occur in vivo^{1, 2}.

In mammalian systems, a specialized form of extracellular matrix (ECM), termed the basement membrane, normally separates epithelial cells from the underlying type I collagen-rich interstitial matrix (1, 2). In mature animals and under physiologic conditions, the epithelium does not establish stable physical contacts with interstitial tissues (1, 2). By contrast, in neoplastic states, transformed epithelial cells (i.e., carcinomas) dissolve the intervening basement membrane barrier and establish adhesive interactions with the newly exposed type I collagen fibrillar network (1-5). As carcinoma cells begin to infiltrate the interstitial matrix, they rapidly adapt themselves to their three-dimensional environment and initiate the proliferative phenotypes that define tumor progression at both primary and metastatic sites (2, 6, 7). Indeed, emphasizing the importance of the tumor-ECM interface, carcinoma cells do not simply use the surrounding interstitial matrix as a passive substrate, they actively promote increased type I collagen deposition within the peri-tumoral microenvironment as a means to further enhance invasive activity, local growth and cancer stem cell formation (7-12).

Despite the importance of the carcinoma cell-type I collagen interface in vivo, therapeutic interventions that directly interfere with the specific cell-ECM interactions operating within this specialized tumor milieu have yet to be identified. Traditionally, new therapeutic agents are developed by identifying a preferred candidate and then generating a specific inhibitor for a targeted effector (13). In this regard, humanized monoclonal antibodies have been established as important players in the therapeutic armamentarium (13, 14). However, strategies that allow for the rapid identification and validation of new targets remain problematic (13). Cogent arguments have been forwarded regarding the utility of phenotypic screens for the purpose of identifying new targets in an unbiased fashion (13, 15). Nevertheless, leveraging this approach requires the engineering of in vitro conditions that faithfully recapitulate carcinoma cell behavior in vivo so that targets can be identified and their functional contribution assessed rapidly prior to in vivo testing.

In view of the state of the art, a need continues to exist for methods and materials useful in identifying therapeutic compositions and compounds that function in vivo.

### SUMMARY

The disclosure provides materials and methods for the discovery, validation, and/or functionalization of unknown molecular biological targets for pharmaceutical intervention and for obtaining specific binding partners, such as antibody products that specifically bind to targets of interest, e.g., cell-surface markers, that are useful therapeutically, prophylactically, and/or diagnostically. Practice of the technology can yield specific binding partners to targets for which efforts to obtain specific binding partners, e.g., cell-surface and non-cell surface binding partners such as proteins, lipids, carbohydrates, and the like elaborated into the cell matrix, such as cytoskeletal proteins, proteases, or autocrine or paracrine factors that may constitute all or part of an antigen, had been unsuccessful to date and can provide increased efficiency and/or efficacy in generating specific binding partners to targets that have been shown to be amenable to the elicitation of specific binding partners. As will be apparent from a review of the entire disclosure, also provided are materials and methods exploiting known molecular biological targets for pharmaceutical intervention and for obtaining specific binding partners, regardless of whether the targets were known to be useful in a particular pharmaceutical intervention, such as diagnosing, treating, preventing or ameliorating a symptom of any of the diseases, disorders or conditions disclosed herein.

The disclosure provides immunogens in a three-dimensional, extracellular matrix that promotes embedded cells to express a unique repertoire of antigens relative to those expressed in standard two-dimensional culture. The three-dimensional extracellular matrix provides a cellular microenvironment that promotes the expression of the unique repertoire of cell-surface proteins by the cells embedded in that microenvironment. As a consequence, the cells present antigenic cell-surface markers that differ from the markers presented by that cell type when present in two-dimensional culture. The difference in cell-surface markers, and hence in antibodies elicited to such markers, is not just a difference without distinction. When injected in vivo, the mouse immune system generates antibodies against cell-surface targets that better recapitulate those antibodies naturally generated in vivo than the antibodies raised against cells in two-dimensional culture. In some embodiments the cell-surface marker is a marker of, or associated with, a disease, such as cancer. Exemplary diseases, disorders or conditions amenable to the disclosed technology include any form of cancer, any form of a fibrotic disease, any form of an inflammatory, cell-mediated, tissue-destructive disease state (e.g., rheumatoid arthritis, giant cell arteritis, Crohn's disease), infectious disease (i.e., disease associated with an infection) or angiogenic disorder (e.g., hypervascularization of cancer tissue, macular degeneration). In some embodiments, the marker is associated with a wound, and an immune response elicited by the three-dimensional presentation of the marker is beneficial in wound healing.

In some embodiments, the cell-surface marker is present on its cognate cell within the three-dimensional environment or framework. The cell-surface marker may also be associated with a portion of a cell surface, such as a cytosolic membrane or it may be engineered such that it is associated with one or more compounds that yield a three-dimensional structure for the cell-surface marker that mimics the structure of the cell-surface marker when found in vivo. Typically, the three-dimensional environment or framework is composed of type 1 collagen (the dominant extracellular matrix protein found in humans) or fibrin (the dominant provisional matrix protein localized to tumor or wound sites).

In some embodiments, the elicitation of specific binding partners, e.g., antibodies, to a cell-surface marker is preceded by a tolerizing step in which the host organism is initially exposed to a three-dimensional environment or framework comprising a normal cell exhibiting cell-surface markers characteristic of that normal cell, e.g., a non-cancerous cell. Following this administration, a three-dimensional environment or framework comprising a cell exhibiting the cell-surface marker of interest, i.e., the target, is administered.

Described with more particularity, the disclosure provides a screening platform wherein human carcinoma cells are cultured within aldimine cross-linked, three-dimensional extracellular matrix protein (e.g., type I collagen) hydrogels similar to those found at invasive sites in vivo (16), and the cancer cell-matrix composite is used to generate a library of monoclonal antibodies (mAbs). In turn, function-blocking or function-activating mAbs are then identified by screening for their ability to suppress carcinoma cell proliferative responses under three-dimensional growth conditions in vitro. Validating the utility of this in vitro approach, selected mAbs are then shown to inhibit carcinoma cell proliferation and metastatic activity in xenograft models in vivo. In addition, employing a combination of immuno-purification, mass-spectroscopy and peptide mapping, the target antigens are identified and their expression confirmed in human cancer tissues. Together, these findings not only establish a platform that allows for the rapid identification of function-blocking or function-activating mAbs and their targets, but also new insights into the regulation of the carcinoma cell-ECM interface within the in vivo setting.

The disclosed materials and methods extend beyond the generation of three-dimensional anti-cancer antigens and methods of screening for antibodies blocking an antigen function involved in cancer development or persistence, such as cell proliferation. Also contemplated are materials and methods for generating three-dimensional antigens of fibrotic disease, an inflammatory disease state, an angiogenic disorder, an infectious disease, or a wound, and methods of treating, preventing or ameliorating a symptom of such a disease, disease state or disorder (e.g., wound) comprising administration of an effective amount of a function-blocking or function-activating antibody according to the disclosure, or a function-blocking or function-activating antibody fragment thereof.

As used herein, an “antibody” is any form of an antigen-binding protein known in the art, including complete immunoglobulin antibodies of any isotype or sub-isotype, a chimera, a humanized or human antibody, an antibody fragment, a scFv, a diabody, a bi-specific antibody fragment, a tri-specific antibody fragment, a fusion protein with any of a wide variety of therapeutic proteins and/or other moieties, a Fab fragment, a Fab′ fragment, a F(ab)2′ fragment and any other functional format for specifically binding an antigen presented in a three-dimensional microenvironment, such as in the hydrogels of the disclosure, or in vivo. Any method known in the art is suitable for producing an antibody product of the disclosure, as defined above. For example, an antibody may be elicited or produced in an immunocompromised recombinant host animal capable of expressing human antibody genes. Alternatively, the antibody may be obtained using an in vitro approach such as phage display, followed by production of the antibody in quantity and, optionally, engineering to form any of the aforementioned antibody products. Alternatively, the antibody may be conjugated to a drug and delivered as an antibody-drug conjugate.

Efforts to develop unbiased screens for identifying novel function-blocking monoclonal antibodies in human carcinomatous states have been hampered by the limited ability to design in vitro models that recapitulate tumor cell behavior in vivo (1,2). Given that only invasive carcinoma cells gain permanent access to type I collagen-rich interstitial tissues, an experimental platform was established wherein human breast cancer cells were embedded in three-dimensional, aldimine cross-linked collagen matrices and used as an immunogen to generate monoclonal antibody libraries. In turn, cancer cell-reactive antibodies were screened for their ability to block carcinoma cell proliferation within collagen hydrogels that mimic the in vivo environment. As a proof-of-principle, one of fifteen function-blocking monoclonal antibodies was further analyzed and demonstrated an ability to halt carcinoma cell proliferation, inducing apoptosis and exerting global changes in gene expression in vitro. The ability of the monoclonal antibody to block carcinoma cell proliferation and metastatic activity was confirmed in vivo and the target antigen identified by mass-spectroscopy as the α₂ subunit of the α₂β₁ integrin, one of the major type I collagen binding receptors in mammalian cells. Validating the ability of the in vitro model to predict patterns of antigen expression in the disease setting, immunohistochemical analyses of breast cancer patient tissues verified markedly increased expression of the α₂ subunit in vivo. These results not only highlight the utility of this discovery platform for rapidly selecting and characterizing function-blocking, anti-cancer monoclonal antibodies in an unbiased fashion, but also identify α₂β₁ integrin as a potential target in human carcinomatous states.

In one aspect, the disclosure provides a method of eliciting an antibody specifically binding a target comprising (a) administering an effective amount of a three-dimensional hydrogel comprising a specific cell type that expresses a biomolecular target molecule; and (b) obtaining an antibody that specifically binds to the target molecule. In some embodiments, the hydrogel comprises type I collagen, fibrin, or a mixture thereof. An exemplary hydrogel comprises type I collagen. Contemplated in most embodiments is the method wherein the type I collagen, fibrin, or a mixture thereof is cross-linked, analogous to the cross-linked state of these molecules in vivo. The disclosure provides methods wherein the biomolecular target molecule is a cell-surface protein, e.g., methods wherein the cell-surface protein is on the surface of a diseased cell. In some embodiments, the diseased cell is a cancer cell, a fibrotic cell, (e.g., fibroblasts, pericytes, mesenchymal stem cells, fibrocytes), an inflammatory cell (e.g., a circulating leukocyte belonging to the neutrophil, eosinophil, mast cell, monocyte/macrophage, or B/T-lymphocyte family), an immune cell (e.g., a neutrophil, a macrophage, a cytotoxic natural killer (NK) cell, a granulocyte, a dendritic cell, a cell from any of various T cell subsets, a B cell) or a cell participating in pathologic angiogenesis, such as an endothelial cell as well as peri-endothelial cell populations (e.g., pericytes, smooth muscle cells or mesenchymal stem cells). Exemplary embodiments include methods wherein the diseased cell is a cancer cell or a fibrotic cell. Also provided are methods wherein the biomolecular target molecule is α2 integrin, α-enolase, calnexin, CD44, filamin, vimentin, or fibrinogen.

For each of the embodiments of this aspect, the disclosure provides methods further comprising a subtractive immunization procedure comprising (a) administering an effective amount of a hydrogel comprising a healthy cell that is a counterpart to, or of the same cell type as, the cell associated with a disease, disorder or condition, to a host organism to elicit an antibody response; and (b) delivering an immunosuppressive agent to the host organism. In some embodiments, the immunosuppressive agent is cyclophosphamide.

In another aspect, the disclosure provides a method of producing an immunogen comprising (a) obtaining a composition comprising a biomolecular target molecule; (b) combining the composition comprising the biomolecular target molecule and a hydrogel-forming compound; and (c) preparing a three-dimensional hydrogel comprising the composition comprising the biomolecular target molecule. In some embodiments, the hydrogel comprises type I collagen, fibrin, or a mixture thereof, and in some embodiments the type I collagen, fibrin, or a mixture thereof is cross-linked. In some embodiments, the composition comprising a biomolecular target molecule is a living cell, such as a diseased cell in a subject such as a human or a non-human animal. Methods according to this aspect are provided wherein the biomolecular target molecule is a cell-surface protein, such as methods wherein the cell-surface protein is on the surface of a diseased cell. Exemplary diseased cells according to this aspect of the disclosure include a cancer cell, a fibrotic cell, a cell involved in pathologic angiogenesis such as an endothelial or peri-endothelial cell involved in pathologic angiogenesis, or a cell involved in a pro-inflammatory disease state such as a leukocyte or blood vessel-associated cell as exemplified by a monocyte (e.g., an M1 macrophage, a dendritic cell, a histiocyte, a Kupffer cell), a granulocyte (e.g., a neutrophil, an eosinophil, a basophil), a T cell, a B cell or a natural killer cell involved in a pro-inflammatory disease state. In some embodiments, the biomolecular target molecule is α2 integrin, a-enolase, calnexin, CD44, filamin, vimentin, or fibrinogen. In some embodiments, the biomolecular target molecule is not known in advance of performing methods according to the disclosure, such as methods of eliciting an antibody or methods of producing an immunogen. By localizing a composition, such as a cell, that comprises a biomolecular target molecule, such as a cell-surface marker, in a three-dimensional hydrogel, the cell is placed in a microenvironment that more closely mimics the in vivo microenvironment and leads to an expression profile that both more closely tracks the expression profile of that cell type in vivo and that differs from the expression profile exhibited by that cell type when cultured in vitro. As a result, a composition comprising a biomolecular target molecule in a three-dimensional hydrogel is a composition, such as a cell, that presents a collection of immunogenic molecules that more closely tracks the molecules presented by that cell type in vivo. The steps involved in generating a composition comprising a biomolecular target molecule in a three-dimensional hydrogel, as disclosed herein, constitute a method according to the disclosure for producing one or more immunogens.

In still another aspect, the disclosure provides a method of identifying an anti-cancer antibody product as functional in vivo comprising (a) contacting a protein capable of cross-linking to form a hydrogel with a cancer cell to produce a seeded hydrogel or hydrogel comprising a cancer cell; (b) incubating the seeded hydrogel or hydrogel comprising a cancer cell; and (c) exposing the seeded hydrogel or hydrogel comprising a cancer cell to an anti-cancer antibody product candidate under conditions suitable for antigen-antibody product binding, wherein binding between the anti-cancer antibody product candidate and the seeded hydrogel or hydrogel comprising a cancer cell identifies the anti-cancer antibody product candidate as an anti-cancer antibody product. The cross-linked protein is a cross-linked matrix protein, such as collagen, e.g., type I collagen, fibrin, elastin, or a mixture thereof. In some embodiments, the extracellular matrix protein contains endogenous aldimine groups to produce the cross-linked protein and/or the protein is modified to generate an aldimine derivative of the protein, thereby allowing the aldimine derivative of the protein to produce the cross-linked protein. Exemplary embodiments are contemplated wherein lysyl oxidase or a transglutaminase catalyzes the modification of the protein to produce the aldimine or iso-peptide derivative of the protein.

In some embodiments of the above-described method, the hydrogel further comprises an α2 integrin holoprotein, such as the α2 β1 integrin. In some embodiments, the hydrogel further comprises the α2 subunit of α2 β1 integrin.

For each of the methods disclosed herein, embodiments are provided wherein the antibody product is a polyclonal antibody, a monoclonal antibody, an antibody fragment, a hybrid antibody, a chimeric antibody, a CDR-grafted antibody, a single chain antibody, a single chain variable fragment antibody, a Fab antibody fragment, a Fab′ antibody fragment, a F(ab′)2 antibody fragment, a linear antibody, a bi-body, a tri-body, a tetrabody, a diabody, a peptibody, a bispecific antibody, a bispecific T-cell engaging (BiTE) antibody, or a chimeric antibody receptor. In some embodiments, the antibody product is a humanized or human antibody product. It will be understood by one of ordinary skill in the art that an antibody product as defined herein also defines an antibody product candidate.

Another aspect according to the disclosure provides an antibody product produced by the method described above, wherein the antibody product is derived from an anti-α2 integrin antibody. Three monoclonal antibodies specifically binding α2 integrin have been obtained, as exemplified by the 4C3 monoclonal antibody. In some embodiments, the antibody product is the 4C3 monoclonal antibody.

Yet another aspect according to the disclosure provides a hydrogel comprising (a) a cross-linked protein; and (b) a biomolecular target molecule such as an integrin protein. Any biomolecular target molecule disclosed herein may be used. In some embodiments, the cross-linked protein is a matrix protein, such as a collagen, e.g., a type I collagen, type III collagen, type IV collagen, fibrin, elastin, hyaluronic acid, laminin, or a mixture thereof. In some embodiments, the integrin protein is α2 β1 integrin and/or the α2 subunit of α2 β1 integrin. In some embodiments, the disclosure provides a hydrogel comprising a cross-linked protein and a biomolecular target, as exemplified by an integrin protein or any protein associated with a disease, disorder or condition of interest. In some embodiments there exists a hydrogel comprising a cross-linked protein and a cell comprising a biomolecular target, e.g., presented on the cell surface, wherein the cell exhibits a disease, disorder or condition. Exemplary cells exhibiting a disease, disorder or condition include a cancer cell, a fibrotic cell, an inflammatory cell, an immune cell, and cells associated with pathologic angiogenesis, such as an endothelial cell, a pericyte, a smooth muscle cell or a mesenchymal stem cell.

Other features and advantages of the disclosure will become apparent from the following detailed description. It should be understood, however, that the detailed description and the specific examples, while indicating some embodiments, are given by way of illustration only, because various changes and modifications within the spirit and scope of the disclosure will become apparent to those skilled in the art from the detailed description.

### BRIEF DESCRIPTION OF THE DRAWING

This patent or application file contains at least one drawing executed in color. Copies of this patent or patent application publication with color drawing(s) will be provided by the United States Patent and Trademark Office upon request and payment of the necessary fee.

FIG. 1. Schematic illustrating MDA-MB-231 breast carcinoma cells, embedded in three-dimensional type I collagen hydrogels (1), used to immunize recipient mice (2). Hybridoma cultures were generated (3) and mAbs tested for their ability to inhibt proliferative responses of MDA-MB-231 cells in three-dimensional culture (4). The abilities of selected mAbs to inhibit MDA-MB-231 proliferative responses were determined in xenograft models in vivo (5) and the antibody targets identified by immunoaffinity isolation and mass-spectroscopy (6).

FIG. 2. Overview of the subtractive immunization procedure.

FIG. 3. MDA-MB-231 bone metastasis model. (A,B) Luciferase-expres sing MDA-MB-231 cells (1×10⁵) were injected into the left ventricle of nude mice with 10 mg/kg of a control IgG1 or mAb 4C3 twice-weekly for 4 weeks and tumor progression monitored by bioluminescent imaging. Representative images shown in (A) were taken at 3 weeks post-injection. Results are expressed as the mean±SEM of control mAb-treated (n=9) and mAb 4C3-treated (n=8) mice. p<0.05. (C) At termination, bioluminescent imaging of spinal fields (black arrow) was assessed with guided X-ray analysis of affected areas in the vertebral column (white arrows) evaluated by microCT. (D) Effect of control IgG1 versus mAb 4C3 on development of hindlimb paralysis during the 4-week treatment period. Results are expressed as the mean±SEM of control IgG1-treated (n=38) and mAb 4C3-treated (n=29) mice. p<0.05.

FIG. 4. Identification of the mAb 4C3 target antigen. (A) mAb 4C3 immunocaptured a 150 kD band from lysates of MDA-MB-231 cells as detected by SDS-PAGE/silver staining. Mass spectrometric sequencing of the band identified the protein as the α2 integrin subunit. (B) MDA-MB-231 lysates were immunoprecipitated with mAb 4C3 and immunoblotted with a second antibody directed against the α2 integrin subunit. (C) Peptide mapping of the mAb 4C3 binding sites in an overlapping series of peptides (10 amino acids each in length) that span the α2 integrin subunit. Asterisks indicate decapeptide epitopes localized within the α-I domain. (D) Schematic illustrating the putative α-I domain elements recognized by mAb 4C3 and 8F10 (labeled “C” and “F”, respectively, within the red circle). Peptide 67 (mAb 4C3 peak) lies within structural element “F” while peptide 44 (mAb 8F10 peak) is located within β sheet “C” the α2 chain as described. The three colored chains (green, yellow, blue) represent a portion of the type I collagen triple helix [model adapted from Emsley et al (29)].

FIG. 5. α2 integrin expression in breast carcinoma bone metastases and primary tissues. Biopsies of breast carcinoma bone metastasis were immunostained for α2 integrin expression in a series of 7 patient samples (three shown here with remaining biopsies presented in FIG. 9). Asterisks mark bone tissue.

FIG. 6. MDA-MB-231 cell adhesion to type I collagen hydrogels was assessed after a one-hour culture period with either a control IgG1 (10 μg/ml), mAb 4C3 (10 μg/ml) or the indicated concentrations of the small molecule α₂β₁ antagonist, TC-I 15 (63). Results are expressed as the mean±SEM (n=3).

FIG. 7. In vitro activity of monoclonal antibody mAb 4C3. (A) MDA-MB-231 cells were seeded in three-dimensional collagen matrices in the absence or presence of mAb 4C3 (10 μg/ml). Cultures were evaluated by phase contrast or confocal microscopy (red) at day 0 and day 4. (B) MDA-MB-231 cells were seeded in three-dimensional collagen in 12-well plates (5×10⁴ cell/well) with mAb 4C3 (10 μg/ml) added at day 0 or day 4 (red arrow). At indicated times, cell number was determined by hemocytometry. Results are expressed as the mean±SEM (n=3). p<0.05. (C) MDA-MB-231 proliferation was assessed by relative ATP levels after 48 hours treatment with indicated mAb 4C3 concentrations. Vehicle controls or control IgG1 mAb were without effect. Adhesion was assessed by allowing MDA-MB-231 cells to attach to collagen gels for 1 hour followed by staining with crystal violet. Results are expressed as the mean±SEM of 3 experiments. (D) Relative levels of caspases 3 and 7 activities were determined for MDA-MB-231 cells embedded within three-dimensional collagen gels for 72 hours in the presence of the indicated concentrations of mAb 4C3 added 24 hr prior to assay. Vehicle control or control IgG1 were without effect. Results are expressed as the mean±SEM of 3 experiments. (E) GO terms identifying cellular processes following mAb 4C3 (10 μg/ml) treatment of three-dimensional-embedded MDA-MB-231 for 48 h. Heat maps of genes regulating cell cycle and apoptosis following mAb 4C3 treatment are shown.

FIG. 8. (A, B) MDA-MB-231 cells (1×10⁵/well) were cultured in 24-well tissue culture plates under two-dimensional conditions in DMEM/10% FCS with or without mAb 4C3 (10 μg/ml) without affecting cell shape at day 3 (A) or proliferation (B). Results are expressed as the mean±SEM of 3 experiments. (C) MDA-MB-231 cells (1×10⁵) were embedded in Matrigel in the presence of a control IgG1 or mAb 4C3 (10 μg/ml each) for 3 days or 4 days without affecting cell shape or cell number. Results are representative of 3 or more experiments.

FIG. 9. (A) Human squamous cell carcinoma (74B; 2×10⁵), ovarian cell carcinoma (ES2; 5×10⁵) or fibrosarcoma (HT1080; 2×10⁵) cell lines were cultured in three-dimensional type I collagen hydrogels for 2 days in the presence of a control IgG1 (10 μg/ml) or mAb 4C3 (10 μg/ml). Phase-contrast micrographs highlight the ability of mAb 4C3 to block cell shape changes. Results are representative of 3 or more experiments. (B) Cell proliferation in three-dimensional collagen was inhibited as a function of mAb 4C3 concentration as assessed by cellular ATP levels with IC₅₀ values reported as the mean±SEM (n=3).

FIG. 10. Anti-carcinoma activity of mAb 4C3 in the chick xenograft model. (A) Vasculature of the chick chorioallantoic membrane (CAM) as visualized following GFP-isolectin B4 (green) infusion by confocal laser microscopy. (B) Perivascular interstitial collagen (blue) in the 11-day-old chick CAM as assessed by second harmonic generation. (C,D) RFP-labeled MDA-MB-231 cells and either mAb 4C3 (0.8 mg/embryo), a vehicle control or control IgG1 (0.8 mg/embryo) were introduced intravenously into the chick embryos. After a 5-day incubation period, tissues were harvested and evaluated by florescent microscopy for presence of MDA-MB-231 cells (orange) and blood vessels (green). Results are representative of 3 or more experiments performed. (E,F) Chick embryos were innoculated i.v. with 2.5×10⁵ luciferase-expressing MDA-MB-231 cells 5 days prior to harvest. Inhibitor, vehicle or control IgG1 was co-administered with the carcinoma cells (day 0) or 24 hours later (day 1). For imaging, eggs were injected i.v. with luciferin 10 minutes prior to retrieval of the lower CAM and imaged for bioluminescence and quantified. Results are expressed as the mean±SEM (n=3). p<0.05.

FIG. 11. Overview of the embryonic chick xenograft model. Shown is a diagram and light microscopic images of the chick embryo and vasculature. Bottom panels show blood vessels (green) and surrounding type I collagen fibrils (blue) as visualized by second harmonic generation microscopy (see also FIG. 10).

FIG. 12. Peptide mapping of the mAb 8F10 binding sites in an overlapping series of peptides (each 10 amino acids in length) that span the α2 integrin subunit. Asterisks indicate decapeptide epitopes localized within the α-I domain.

FIG. 13. α₂ integrin staining of four additional biopsy specimens of human breast cancer patients with bony metastases. Asterisks indicate bone, and arrows indicate metastatic breast carcinoma cells.

FIG. 14. Breast tissue biopsy specimens harvested from primary sites highlighting strong α₂ expression in breast carcinoma tissues (black arrows) with additional, but weaker, staining outlining normal myoepithelial cells. In Case 1, the tumor embolus as well as the lymphatic endothelium are positive for α₂ expression.

FIG. 15. Characterization antibodies recognizing integrin subunit α2. A. Complementarity Determining Regions (CDR) for mAbs 4C3, 8F10 and 2D11. B. Proposed common epitope for 4C3, 8F10 and 2D11 within the α-I domain of integrin subunit α2. Crystallography figure from Emsley et al., Cell 101, 47, 2000.

FIG. 16. Luciferase-expressing MDA-MB-231 cells (5×10⁶) were orthotopically injected into nude mouse recipients and either vehicle 250 μg mAb 4C3/mouse (about 10 mg/kg) or 500 μg mAb 4Ce/mouse (about 20 mg/kg) given i.p 3 times weekly, and tumor volume and luminescence monitored as described. Results are expressed as the mean±SEM (n=4).

FIG. 17. Immunohistochemistry with mAb 4C3. A human breast tumor tissue array was stained with mAb 4C3 (brown) and nuclei counterstained with hematoxylin (blue).

### DETAILED DESCRIPTION

The disclosure provided herein is based, in part, on the realization that medically relevant biomolecular target molecules are frequently found in association with cells in the in vivo environment. Such associations may affect the spatial presentation of such targets, such as cell-surface protein markers, lipoproteins, nucleoproteins, glycoproteins or, indeed, any biomolecule capable of serving as a target. A major approach to the identification or recognition of a particular biomolecular target is an immunological approach in which an antibody that specifically binds to a target is elicited and subsequently used in medically relevant procedures such as diagnosis, prophylaxis, therapy or amelioration of a symptom of a disease, disorder or condition. Immunological approaches have been developed and verified over the past few decades such that there now exist many forms of antibody products that retain the binding specificity of a parent antibody but differ from that antibody in ways explained more fully below. The tremendous power of immunology to provide valuable diagnostic, prophylactic and therapeutic tools, however, is limited by the availability of antigens that accurately reflect a biomolecular target molecule as it exists in vivo.

The technology disclosed herein takes an unusual approach to antibody elicitation in not seeking to purify a target molecule so as to maximize the likelihood of identifying a target-specific antibody in a conventional antibody screen; rather, the technology retains the target molecule in its complex, natural, in vivo-like, three-dimensional cellular environment to maximize the likelihood that a target-specific antibody, when identified, will also specifically recognize the target in vivo. Moreover, the repertoire of expressed genes and gene products are completely different in standard 2-dimensional culture conditions compared to the three-dimensional microenvironments disclosed herein. The cells present on their surface numerous proteins, and the composition of the cell-surface proteins depends on the extracellular environment of that cell. Recognizing that this approach may elicit a greater variety of antibodies than conventional approaches, also disclosed herein is a method of tolerizing antibody-generating organisms to reduce the presence of undesired antibodies recognizing a cellular antigen that is found on normal cells, and therefore not of interest. For example, the likelihood of identifying an antibody recognizing a cancer-specific target molecule is enhanced by first exposing the antibody-generating host organism to a healthy cell of the same type as the cancer cell, and then eliminating antibody-producing cells responding to the healthy cells prior to challenge with the cancer cell. This optional tolerization step reduces the complexity in identifying a target-specific antibody while retaining the advantage of using a form of the antigen of interest that mimics its form in vivo, thereby enhancing the opportunity to identify and develop medically useful antibody products.

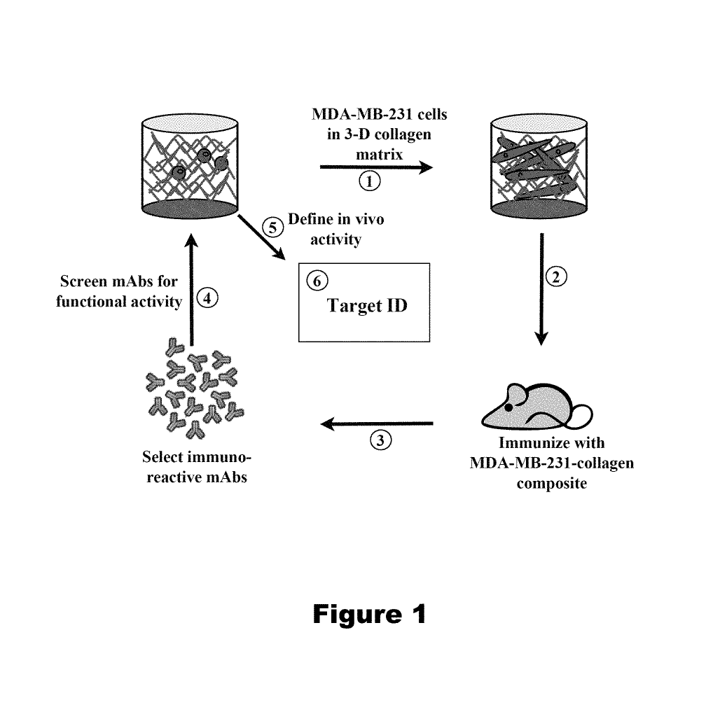

Recently developed and disclosed herein are model systems wherein key aspects of cancer cell behavior observed in vivo can be mimicked in vitro. This experimental hurdle has been negotiated, at least in part, by embedding cells in three-dimensional extracellular matrices whose major components and structural organization closely match those encountered at primary and metastatic sites in vivo. To incorporate advances in tumor cell culture techniques into a high-throughput screening paradigm that enables selection of targets in an unbiased fashion, well-characterized, human carcinoma cell lines or primary human carcinoma stem cells have been established in three-dimensional extracellular matrices that have been constructed from type I collagen, the most abundant ECM molecule found in humans, or fibrin, the blood clotting protein found surrounding cancer cells at all neoplastic sites (16, 96)] and used as immunogens to generate panels of monoclonal antibodies (FIG. 1).

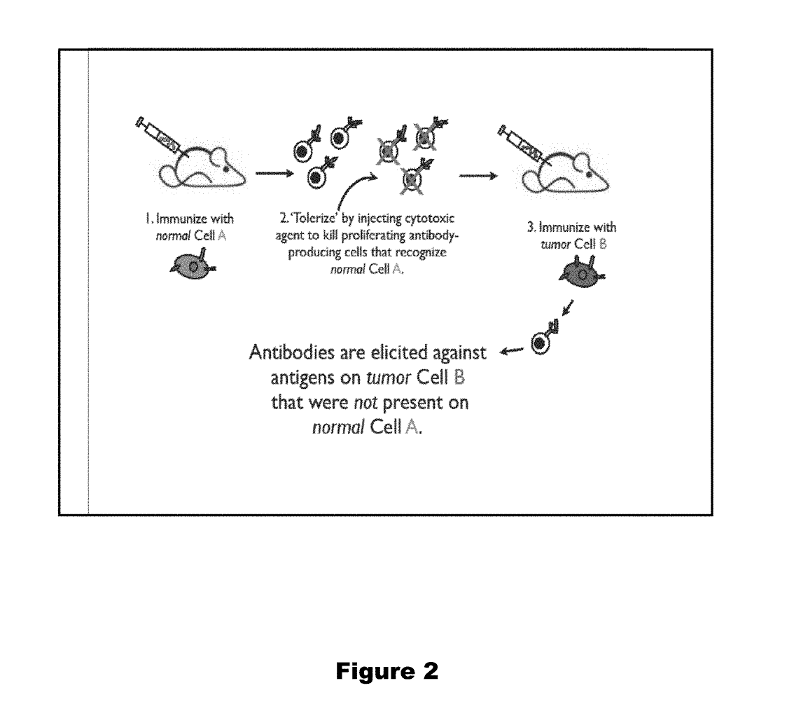

Insuring that the elicited immune response is restricted to the cancer cell populations, the collagen or fibrin hydrogels are constructed from mouse proteins, and the generated panels of monoclonal antibodies are then screened for those that recognize the intact tumor cells by ELISA. Positive clones are then expanded and further screened for functional activity as defined by their ability to inhibit cancer cell invasion or growth in three-dimensional microenvironments. Monoclonal antibodies demonstrating anti-cancer activity represent potential therapeutic agents in their own right and can be used (following immunopurification and mass-spectroscopy) to identify molecular targets of demonstrable utility. To further enrich for tumor-specific antigens, an immunological technique known as subtractive immunization was also used, as described in Example 7 and illustrated in FIG. 2.

In the subtractive immunization procedure, mice are immunized with the normal or healthy counterpart of the human carcinoma cells (e.g., in the case of breast cancer, animals are primed with normal or healthy human mammary epithelial cells) and then treated with the immunosuppressive agent, cyclophosphamide. These mice prevented from maintaining an immune response against antigens found on the normal human epithelial cells, termed ‘tolerized’ mice, are then challenged by injection of human carcinoma cells. This experimental protocol results in an enhanced immune response directed toward antigens found specifically on the tumor cells.

To date, the standard immunization protocol using three-dimensional collagen-cancer cell composites (i.e., three-dimensional immunogens) has been applied to at least five types of cancer, i.e., a breast cancer [using the MDA-MB-231 cell line (17) as well as the stem cell-enriched, breast carcinoma line, SUM159 (16)], primary human glioblastoma stem cells, pancreatic carcinoma cells, melanoma cells and ovarian carcinoma cells. As noted in Table 1, to date approximately 300 monoclonal antibody-generating hybridoma lines that recognize one or more of these cancer cells have been generated.

The technology disclosed herein provides the materials and methods for rapidly and reproducibly generating specific binding partners to any of a wide variety of molecular targets, such as cell-surface markers found on cells characterized by a disease, disorder or condition. Exemplary diseases, disorders or conditions include a cancerous condition, a fibrotic condition, a hypervascularized condition or a pro-inflammatory condition. Without wishing to be bound by theory, the technology maximizes the efficiency and efficacy of eliciting specific binding partners (e.g., antibody products) to target molecules of interest by mimicking the in vivo environment of a host organism, such as a human subject or patient, having the disease, disorder or condition. Towards that end, the technology provides cells characterized by the disease, disorder or condition in the three-dimensional environment of a hydrogel also comprising type 1 collagen and/or fibrin, and/or elastin. Typically, the cell presents the target molecule of interest in the form of a cell-surface marker, such as a cancer marker, a marker of fibrotic disease, a marker of pathologic angiogenesis or a marker of pro-inflammatory disease. In some embodiments, elicitation of the specific binding partner, such as an antibody product, is preceded by exposing the host organism to a tolerization step involving administration of a healthy cell otherwise similar or identical to the diseased cell.

The disclosed technology will be better understood after considering the features of that technology described below.

Target Molecules

Any biomolecular target molecule known or reasonably believed to be involved in a biological process implicated in a disorder, condition or disease state, and any unknown biomolecular target molecule found in a microenvironment characterized by a disorder condition or disease state (e.g., a diseased cell), is embraced by the technology disclosed herein. Target molecules may be proteins or peptides, or nucleic acids such as RNA, DNA, or a non-naturally occurring nucleic acid, or lipids, or any other biomolecule capable of contributing to an antigenic determinant specifically recognized by at least one vertebrate antibody. Further, the target molecule may be a fused molecule, such as would be found in lipoproteins and nucleoproteins. The target molecule may also be derivatized, e.g., a glycosylated protein or a phosphorylated protein. Typically, a suitable biomolecular target is bound or associated with the surface of at least one cell type.

One advantage of the technology disclosed herein is that in vitro, three-dimensional microenvironments may comprise, e.g., cells that, in turn, comprise one or more unknown biomolecular targets that are used in preparing the three-dimensional immunogens used to elicit target-specific antibodies functional in vivo. In some embodiments, however, the technology embraces in vitro, three-dimensional microenvironments comprising cells that, in turn, comprise one or more known biomolecular targets, such as α-fetoprotein (AFP) , CA15-3, CA27-29, CA19-9, CA-125, Calcitonin, Calretinin, Carcinoembryonic antigen, CD34, CD99MIC 2, CD117, Chromogranin, Cytokeratin (various types), Desmin, Epithelial membrane antigen (EMA), Factor VIII, CD31 FL1, Glial fibrillary acidic protein (GFAP), Gross cystic disease fluid protein (GCDFP-15), HMB-45, Human chorionic gonadotropin (hCG), inhibin, keratin (various types), MART-1 (Melan-A), Myo D1, muscle-specific actin (MSA), neurofilament, neuron-specific enolase (NSE), placental alkaline phosphatase (PLAP), PTPRC (CD45), S100 protein, smooth muscle actin (SMA), synaptophysin, thyroglobulin, thyroid transcription factor-1, Tumor M2-PK, and vimentin. Exemplary fibrotic cell markers include α2 macroglobulin, α2 globulin (or haptoglobin), γ globulin, apolipoprotein A1, γ glutamyltranspeptidase, and bilirubin. Exemplary fibrotic cell targets include Plasminogen activator inhibitor-1 (PAI-1); Alpha-2-macroglobulin; Alpha-crystallin B chain; Decorin; Four and a half LIM domains (Fhl2); Major prion protein (CD230) (RaPrP); Alpha-1, type 1Collagen; Smooth muscle aortic alpha-actin; Beta-tropomyosin (TPM2); Collagen, type XII, alpha-1 (Coll2a1); Secreted phosphoprotein 1 (Spp1); Lectin, galactose binding, soluble 1 (Lgals1); Phosphoprotein enriched in astrocytes 15 (Pea15); Transgelin (Tagln); Lipoprotein lipase (Lpl); Matrix Gla protein (Mgp); Troponin T2, cardiac (Tnnt2); Glypican 3 (GPC3); Glutathione peroxidase 3 (Gpx3); Similar to Lox1 protein (LoxL1); Lysyl oxidase (Lox); Small inducible cytokine subfamily D, number 1 (CX3CL1); Lumican (Lum); and Cytochrome P450, family 1, subfamily b, polypeptide 1 (Cyp1b1).

The above-identified target molecules are suitable for use in methods according to the disclosure, but it is not necessary to identify a target molecule in advance of efforts to elicit a specifically binding antibody. An advantage of the disclosed technology over known methodologies is that the entire cell giving rise to or participating in the disease, disorder or condition is typically used to elicit an antibody response. Subsequent screens may be performed to eliminate antibodies binding to targets present on both healthy and diseased cells of a given type, such as “housekeeping” markers. As an alternative to post-elicitation screens, a tolerization step can be added to the elicitation protocol to reduce or eliminate antibodies specifically binding to targets found on both healthy and diseased cells of a given type. As examples, an immunogen could be additional cells (e.g., cancer-associated fibroblasts, monocytes, T cells (e.g., CTLs, Tregs)); additional factors (e.g., cytokines, growth factors); additional manipulations (transfection or gene targeting in the cells included in the immunogen, e.g., cells with mutated K-Ras following subtractive immunization with wild-type Ras cells); or altered conditions (e.g., hypoxia or altered media conditions).

Importantly, the disclosure provides for a three-dimensional microenvironment comprising, typically, a cell representative of cells useful in diagnosing a disease, disorder or condition, a cell useful in preventing or treating a disease, disorder or condition, or a cell providing a target useful in obtaining a target-specific binding partner such as an antibody product according to the disclosure. Thus, it can be seen that methods according to the disclosure use cells comprising a target biomolecule that may either be known in the art or unknown in the art. The disclosed methods are useful in selecting specific binding partners that recognize and bind to the form that a target assumes in vivo, but the methods are also useful in providing methods for obtaining target-specific antibody products that specifically bind to target biomolecules and exert a biologic effect (e.g., function-blocking, function-enhancing or capable of triggering an immune response) that were never identified as having any association to a particular disease, disorder or condition.

Cells

Consistent with the discussion on target molecules, cells embraced by the disclosure include any cell type that causes or manifests a disease, disorder or condition that one would like to prevent, diagnose or treat, or for which symptom amelioration is desired. A wide variety of healthy cell types can change to give rise to or exhibit a disease, disorder or condition, and the disclosed technology embraces such cells. Exemplary cells include any cell type capable of existing in a cancerous state or giving rise to a cancer, such as Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer in Adolescents, Cancer in Children, Cancer in Young Adults, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Leukemia—Acute Lymphocytic (ALL) in Adults, Leukemia—Acute Myeloid (AML), Leukemia—Chronic Lymphocytic (CLL), Leukemia—Chronic Myeloid (CML), Leukemia—Chronic Myelomonocytic (CMML), Leukemia in Children, Liver Cancer, Lung Cancer, Lung Cancer—Non-Small Cell, Lung Cancer—Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, Wilms Tumor, or Head and Neck Squamous Cell Carcinoma.

Additional exemplary cells include any cell types capable of giving rise to or participating in Pulmonary fibrosis, Idiopathic pulmonary fibrosis, Cystic fibrosis, fibrosis of the liver, Cirrhosis of the liver, Endomyocardial fibrosis, Old myocardial infarction, Atrial Fibrosis, Mediastinal fibrosis, Myelofibrosis, Retroperitoneal fibrosis, Progressive massive fibrosis (lungs), Nephrogenic systemic fibrosis (skin), Crohn's Disease, Keloid (skin), Scleroderma/systemic sclerosis (skin, lungs), Arthrofibrosis (knee, shoulder, other joints), Peyronie's disease (penis), Dupuytren's contracture (hands, fingers), or some forms of adhesive capsulitis (shoulder). Other exemplary cells include cells involved in pathologic angiogenesis, such as endothelial cells, pericytes, smooth muscle cells or mesenchymal cells, as well cells involved in pro-inflammatory disease states such as any of the various leukocyte cell populations (e.g., hematopoietic stem cells, myeloid leukocytes such as monocytes, macrophages and granulocytes (e.g., neutrophils, eosinophils, and basophils), and lymphocytes such as T cells, B cells and natural killer cells).

Another group of exemplary cells include cell types involved in inflammatory processes associated with a disease, disorder or condition, including but not limited to, cell types giving rise to or participating in Alzheimer's disease, ankylosing spondylitis, appendicitis, arthritis (including osteoarthritis, rheumatoid arthritis (RA), and psoriatic arthritis), autoimmune diseases (including rheumatoid arthritis (RA), systemic lupus erythematosus (SLE)), asthma, atherosclerosis, bursitis, cancer (e.g., gallbladder carcinoma), colitis, complex regional pain syndrome, Crohn's disease, cystitis, dermatitis, diverticulitis, fibromyalgia, hay fever, hepatitis, inflammatory myopathies, irritable bowel syndrome (IBS), nephritis, Parkinson's disease, periodontitis, phlebitis, reflex sympathetic dystrophy, reflex neurovascular dystrophy, rhinitis, tendonitis, tonsillitis, ulcerative colitis, and vasculitis.

Apparent from the disclosure, there are numerous groups of cells that can be incorporated into the three-dimensional immunogens alone or in combination (e.g., cancer cells with endothelial cells or mesenchymal stem cells) disclosed herein and which, in a diseased state in vivo, can be the focus of the immunologically based diagnosis, prevention, treatment or symptom amelioration methods according to the disclosure. One further category of exemplary cells are the cells of the vasculature, such as endothelial cells, that are involved in pathologic angiogenesis.

Microenvironment

Another feature of the disclosed technology is the microenvironment providing context for the (typically) cell-based target molecules used as antigens and as screening tools to identify specifically binding antibodies and antibody products. The in vitro, three-dimensional microenvironment mimics the in vivo environment of the target-containing entity (e.g., a cell presenting the target on its surface, a macromolecular complex comprising the target) in at least one important aspect. Typically, the microenvironment contains an ECM protein with which the target-containing entity is associated in vivo, such as a type I collagen matrix or a fibrin matrix. These matrices may have a single extracellular protein or a mixture of such proteins. Additional compositions that may be found in a microenvironment include any compound found associated with the ECM in vivo, such as type III or type IV collagen, elastin, hyaluronic acid and/or laminin.

Three-Dimensional Immunogens

Apparent from the disclosure is the fact that the target molecules used to elicit specifically binding antibodies, whether those target molecules have been identified before elicitation or not, are provided to the antibody-generating organism in a three-dimensional microenvironment that mimics the three-dimensional environment in which the target molecules are found in vivo. Typically, two levels of mimicry are used to maximize the resemblance of the target molecule used as immunogen to the target molecule found in vivo. The first level of mimicry typically involves locating the specific target in its normal in vivo cellular microenvironment, such as by locating a cell-surface biomolecular target on the surface of the cell where it is found in nature, or locating the a biomolecular target in a microenvironment comprising a macromolecular complex for targets naturally found in such microenvironments. For cell-associated biomolecular targets, a second level of mimicry involves the typical placement of the cell within an ECM-like microenvironment that mimics the in vivo microenvironment of the cell, such as the ECM. In using this three-dimensional approach to antigen preparation, the disclosed technology maximizes the likelihood that any specific binding partner elicited in an antibody-generating organism will also recognize the target in its in vivo environment. This significantly increases the likelihood of eliciting, and if desired, constructing a specific binding partner of medical value in diagnosis, prophylaxis, treatment or amelioration of a symptom of a disease, disorder or condition.

Antibodies and Antibody Products

The technology does not limit the type (isotype or sub-isotype) of an antibody elicited using an immunogen according to the disclosure, and the technology does not limit the ultimate form of antibody product that may be derived from such an antibody for use in any of the diagnostic, prophylactic, therapeutic, or symptom-amelioration methods disclosed herein. In addition, the technology embraces antibody products derived from antibodies elicited in any host organism known in the art, including any vertebrate species, such as man, any domesticated animal or any laboratory animal, e.g., mouse, rat, goat, sheep, cat, dog, horse, or cattle, and camelid antibodies. Moreover, the disclosure contemplates antibody products derived from antibodies identified from libraries that are screened in three-dimensional ECM hydrogels in vitro, such as by using phage screening technologies.

The antibody may be any type of immunoglobulin known in the art. In exemplary embodiments, the antibody product is derived from an antibody of isotype IgA, IgD, IgE, IgG, or IgM. Also, the antibody product in some embodiments is a monoclonal antibody or is derived from a monoclonal antibody. In other embodiments, the antibody product is a polyclonal antibody or is derived therefrom. In some embodiments, the antibody product is derived from an antibody that is a naturally occurring antibody, e.g., an antibody isolated and/or purified from a mammal, or produced by a hybridoma generated from a mammalian cell.

Methods of producing antibodies are well known in the art. In some embodiments, the antibody product is a genetically engineered antibody, e.g., a single-chain antibody, a humanized antibody, a chimeric antibody, a CDR-grafted antibody, a human engineered antibody, a bispecific antibody, a trispecific antibody, and the like. Genetic engineering techniques also provide the ability to make fully human antibodies in a non-human source. In some aspects, the antibody product is in polymeric, oligomeric, or multimeric form. In certain embodiments in which the antibody product comprises two or more distinct antigen binding region fragments, the antibody product is considered bispecific, trispecific, or multi-specific, or bivalent, trivalent, or multivalent, depending on the number of distinct epitopes that are recognized and bound by the antibody product.

In some aspects according to the disclosure, the antibody product is an antigen binding fragment of an antibody. The antigen binding fragment, or portion, may be an antigen binding fragment of any of the antibodies or antibody products described herein, provided that the fragment retains the specific binding property of the whole antibody. The antigen binding fragment can be any part of an antibody that has at least one antigen binding site, including but not limited to, a Fab, a Fab′, a F(ab′)₂, a dsFv, a sFv, a scFv, a diabody, a triabody, a tetrabody, a bispecific T-cell engager or BiTE, a bis-scFv, a fragment expressed by a Fab expression library, a domain antibody, VhH domains, V-NAR domains, a VH domain, a VL domain, and the like.

Kits

In another aspect, a kit is provided that comprises a compound suitable for use in preparing a hydrogel, such as type 1 collagen or fibrin, or both compounds, a pharmaceutically acceptable adjuvant, diluent or carrier, and a protocol for preparation and administration of an immunogen according to the disclosure.

Prevention, Prophylaxis or Vaccine; Treatment; Diagnosis

The disclosure provides a new approach to harnessing the power of the immune system to combat diseases, disorders and conditions in a general sense. Accordingly, a wide variety of diagnostic, prophylactic, therapeutic, and symptom-ameliorating methods are provided to administer the antibody products useful in detecting and/or modifying an activity of any of the wide range of target molecules immunologically detectable and suitable for incorporation into the immunogens according to the disclosure.

An exemplary family of diseases amenable to diagnosis, prophylaxis, or therapy according to the disclosure is the group of cancer diseases. Cancers associated with any of the cancer cells identified in the section addressing cells (see above) are contemplated as suitable for diagnosis, prophylaxis, or treatment using antibody products elicited using three-dimensional immunogens comprising at least one such cancer cell. In diagnostic methods, known cancer cell-surface markers are identified by antibody products ultimately elicited using the marker in a microenvironment mimicking its in vivo environment. Vaccines are also contemplated that comprise a hydrogel comprising a cell presenting a biomolecular target molecule of the disclosure. Such vaccines will elicit at least one antibody product that specifically binds to a biomolecular target molecule functionally involved in elaboration of a relevant disease process, triggering an immune response against the target molecule. Treatment methodologies are also provided wherein a target molecule functionally involved in disease progression is bound by an antibody product elicited according to the disclosure and wherein the bound target molecule is inhibited or prevented from providing the function relevant to disease progression. In related methodologies, an antibody product specifically binds to a target molecule involved in the presentation of a symptom of a disease, disorder or condition and the specific binding of the antibody product inhibits or prevents the target molecule from providing the function involved in symptom presentation, thereby ameliorating a symptom of a disease, disorder or condition.

Other exemplary diseases, disorders or conditions include fibrosis in any of its known forms, pathologic angiogenesis and pro-inflammatory disease states. For each disease, disorder or condition, the disclosure comprehends methods of diagnosis, methods of prevention or prophylaxis, methods of treatment, and methods of ameliorating at least one symptom of the disease, disorder or condition.

Describing the aspects of the disclosure in greater detail, recent interest has focused on designing unbiased phenotypic screens wherein the identification of function-blocking effects precede efforts to dissect the underlying molecular mechanisms that give rise to the desired outcomes. With increasing evidence that cell behavior in three-dimensional culture systems more faithfully recapitulates in vivo function, greater emphasis has been placed on developing improved in vitro models for screening purposes, including the use of basement membrane-like gels, pepsin-extracts of dermal collagen and synthetic hydrogels (1, 2, 6, 33-35). However, the degree to which any of these constructs recapitulate the structure or function of the native ECM deposited in vivo remains controversial (1, 2, 16, 34). In carcinomatous states, neoplastic cells at both primary and metastatic sites are known to interface a network of covalently cross-linked type I collagen fibrils that have physical properties that modulate tumor phenotypes (2, 3, 5-12, 16). As such, type I collagen hydrogels that are naturally cross-linked by lysyl oxidase-derived aldimine bonds (16) to promote carcinoma cells to express a more in vivo-like display of surface antigens were selected and these hydrogels could be used both as an immunogen for monoclonal antibody (mAb) production as well as a physical platform for functional screening.

Functional screening is often used to identify antibody products that target a given antigen, such as a tumor antigen. The antibody product, including antibodies or antibody fragments, can be any form of antibody known in the art, such as a full-length polyclonal antibody or a full-length monoclonal antibody. Antibody fragments according to the disclosure retain at least one specific binding characteristic of the parent whole antibody. An antibody according to the disclosure can be derived from any class, such as an immunoglobulin G or IgG antibody, and can be of any sub-class, such as an IgG1, IgG2, IgG3, or IgG4 antibody. The antibody can be a humanized or human antibody, a chimeric antibody, or a CDR-grafted antibody. Moreover, an antibody fragment according to the disclosure comprises the antigen binding site of the parent antibody and includes, e.g., a Fab fragment, a Fab′ fragment, a F(ab′)2 fragment, a single-chain antibody, a single-chain Fv (i.e., scFv) molecule, a linear antibody, a diabody, a peptibody, a bi-body (bispecific Fab-scFv), a tribody (Fab-(scFv)2), a hinged or hingeless minibody, a mono- or bi-specific antibody, and antibody fusion proteins comprising the antigen binding site of the parent antibody. Additionally, the antibody or antibody fragment as described above may further comprise a second polypeptide covalently bound to the antibody or antibody fragment in a fusion polypeptide, for example an antibody or antibody fragment described above wherein the second polypeptide is a cytotoxic polypeptide. The antibody or antibody fragment may also be associated with a non-proteinaceous cytotoxin. In some embodiments, the antibody or antibody fragment is labeled. The antibody (or fragment) may also contain a sequence conferring additional properties, such as a cellular import function (e.g., Trans Activator of Transcription (TAT) or the HSV70 co-chaperone known as Coat Protein Interacting Protein (CPIP) fusion). The antibody or fragment may be labeled or bar-coded.

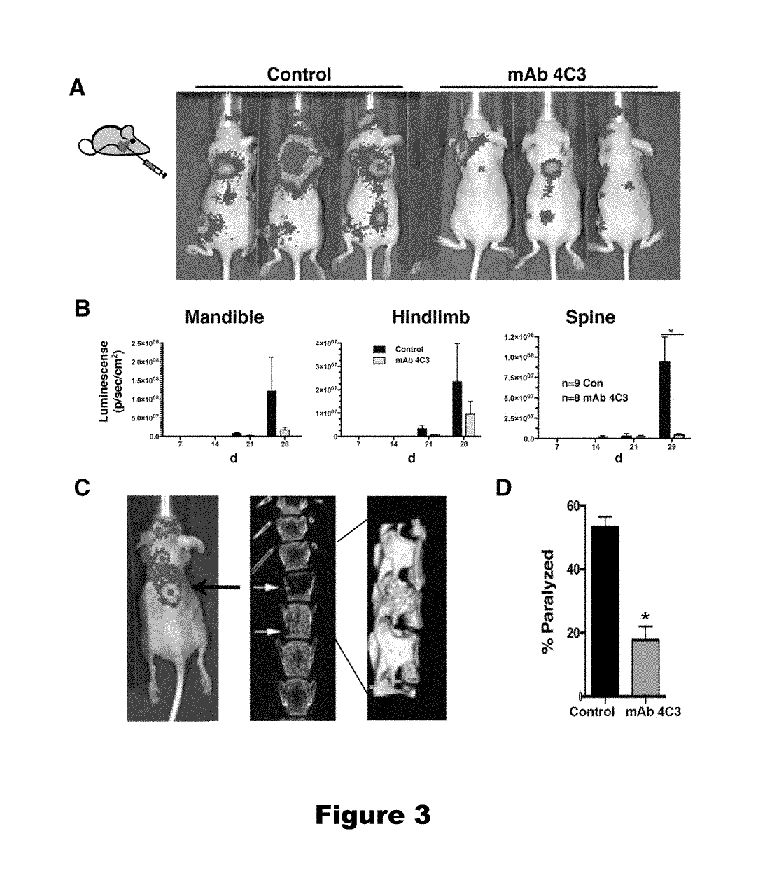

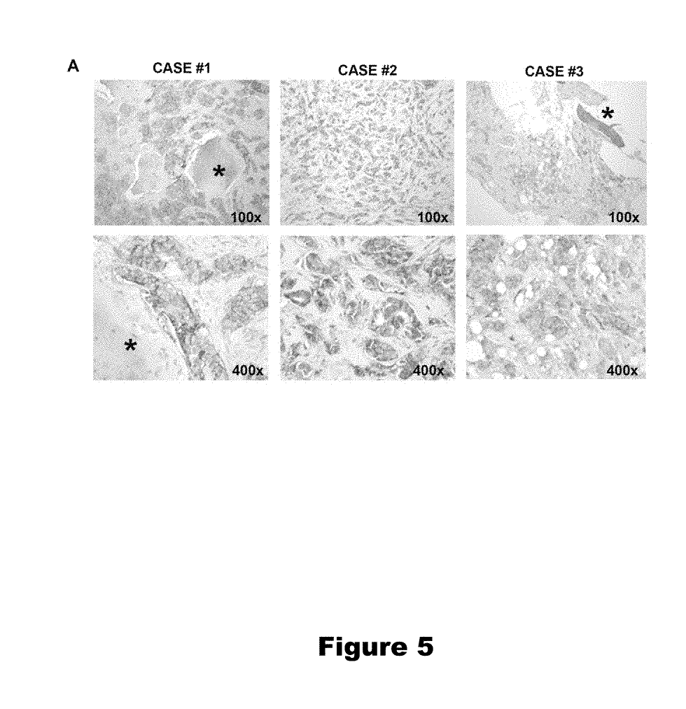

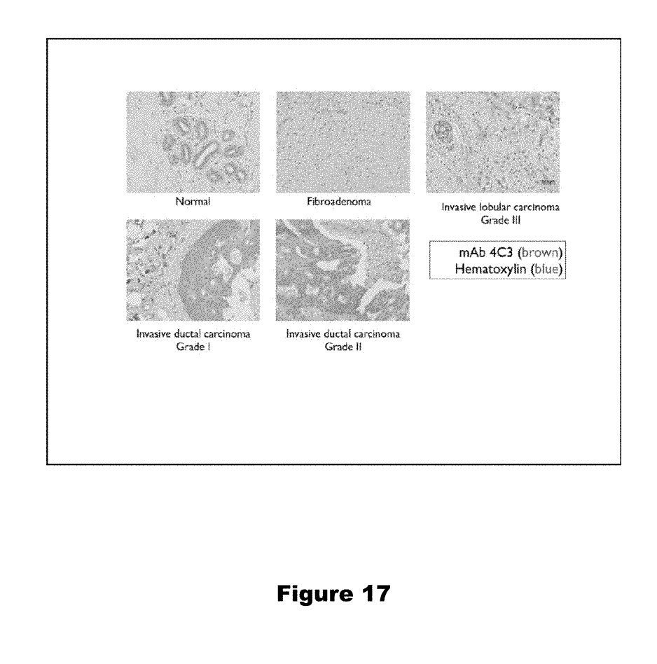

Using the experimental approach noted above, approximately 5% of the generated monoclonal antibodies (mAbs) displayed growth-inhibitory effects. The monoclonal antibody designated mAb 4C3 was selected for additional analysis based on its inhibitory activity in our in vitro screen using target cells embedded in three-dimensional type I collagen hydrogels. Importantly, antibody 4C3 did not exert any growth inhibitory effects in standard two-dimensional culture. Based on these results, antibody 4C3 was further characterized as a proof-of-principle prototype to determine whether i) function-blocking activity detected initially in vitro could be extended into in vivo settings, ii) the mAb-reactive antigen could be identified and iii) target antigens discovered using human carcinoma cell-type I collagen composites faithfully predict in vivo patterns of expression in patient samples. As described in Example 4 and shown in FIGS. 5 and 17, mAb 4C3 successfully inhibited the perivascular proliferation of extravasated MDA-MB-231 cells within the three-dimensional type I collagen-rich interstitial matrix of the live chick embryo, an in vivo model xenograft system wherein cancer cell behavior, including invasion, proliferation and metastasis recapitulate those observed in mouse xenograft models (22, 23). Further, the utility of mAb 4C3 to inhibit MDA-MB-231 proliferation in a mouse model was assessed wherein the cancer cells were allowed to metastasize to mouse skeletal tissues, a type I collagen-rich environment relevant to the bone metastatic activity displayed in human patients (17, 26-28). While effects of mAb 4C3 on carcinoma growth within the mandible and hindlimb supported mAb-mediated inhibitory effects, MDA-MB-231 proliferation in the vertebral column was almost completely inhibited, with significant effects on the development of paralysis-associated morbidity (FIG. 3).

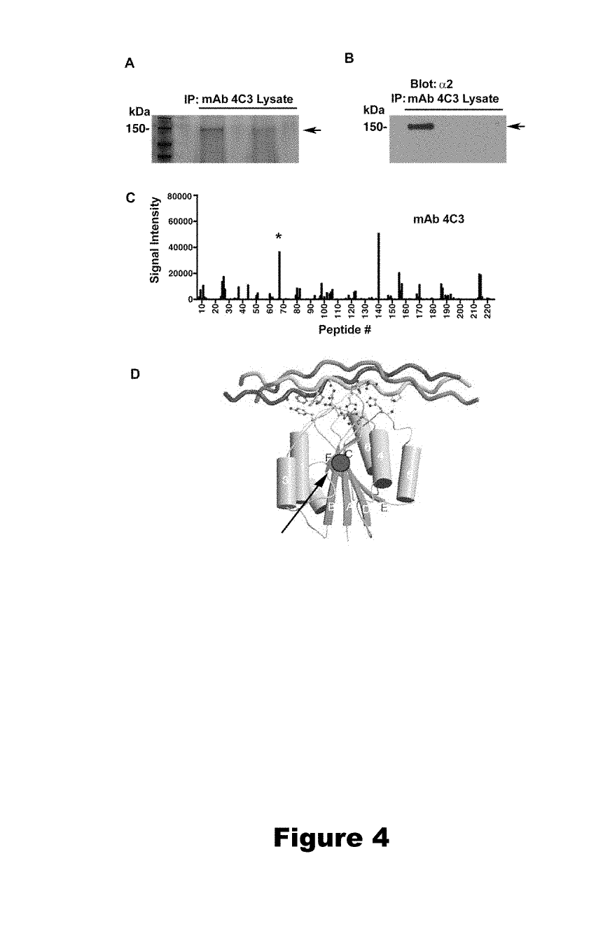

Following immuno-affinity purification and mass spectroscopy, the mAb 4C3 target antigen was identified as the α₂ integrin subunit, whose only known partner, the β₁ integrin chain, forms a heterodimeric complex that serves as a major type I collagen-binding receptor (29, 30) (FIG. 4). Peptide mapping characterized the mAb 4C3 epitope within the α-I domain of the α₂ integrin, a metal ion-dependent adhesion site that is responsible for ligand recognition and binding (29, 30) (FIG. 4). While these results dovetail a number of reports documenting important roles for α₂β₁ in mediating cancer cell-type I collagen interactions in vitro, ranging from proliferation and invasion to epithelial-mesenchymal transition and cancer stem cell formation (36-48), the function of the α₂ integrin in neoplastic states in the in vivo setting is less clear. Recently, Ramirez et al concluded that α₂β₁ serves as a metastasis suppressor in mouse models as well as human cancer (49). Using α₂ integrin-null mice that were bred into a mouse mammary tumor virus-Neu transgenic line, they demonstrated that despite the complete absence of α₂β₁, tumor initiation was only marginally affected while lung metastatic activity was actually enhanced (49). However, in this mouse model, all tissues are rendered α₂ integrin-deficient throughout embryonic and postnatal development. Hence, the MMTV-Neu oncogene is expressed, by necessity, in α₂ integrin-null mammary epithelial cells where potential effects of the integrin on tumor transformation and progression are difficult to define (i.e., as opposed to deleting the α₂ integrin in committed carcinoma cells). Indeed, in contrast to these findings, targeting α₂β₁ with either function-blocking antibodies or shRNA-based strategies has been reported to block metastatic activity in a number of animal model systems (50-53). Likewise, in a second in vivo model of cancer progression using α₂-null mice bred into a K14-HPV16 transgenic line, squamous carcinoma cell proliferation and metastatic activities were decreased in the absence of the α₂ integrin (54).

Independent of studies in mouse models, recent studies of human breast cancer and prostate cancer samples indicate that α₂ mRNA expression levels can decrease as a function of increased metastatic burden and decreased survival (49). However, at the protein level, α₂β₁ is readily detected at both primary and metastatic sites in a variety of cancers, including breast (as described herein; FIG. 5) and prostate cancer (52, 55, 56). While it may be reasonable to conclude that high levels of α₂β₁ can potentially retard motile responses by promoting adhesion, lower levels of the integrin may nevertheless be required to support the cell-ECM interactions most conducive to invasion and growth. Nevertheless, it is unlikely that all carcinomas will prove equally dependent on α₂β₁ as other collagen-binding adhesion molecules, including α₂β₁, α₁₀β₁, α₁₁β₁ and discoidin receptors, have been described (30). As such, it should be stressed that the intent of using carcinoma cell-type I collagen composites as an antigen for mAb production is not to simply identify collagen-binding ligands, but rather to generate mAbs that interfere with cancer cell behavior in an environment similar to that encountered in vivo. Indeed, these studies indicate that most of the function-blocking mAbs identified in screens performed to date do not target type I collagen receptors (see below), but rather surface molecules with as yet to be characterized mechanisms of action.

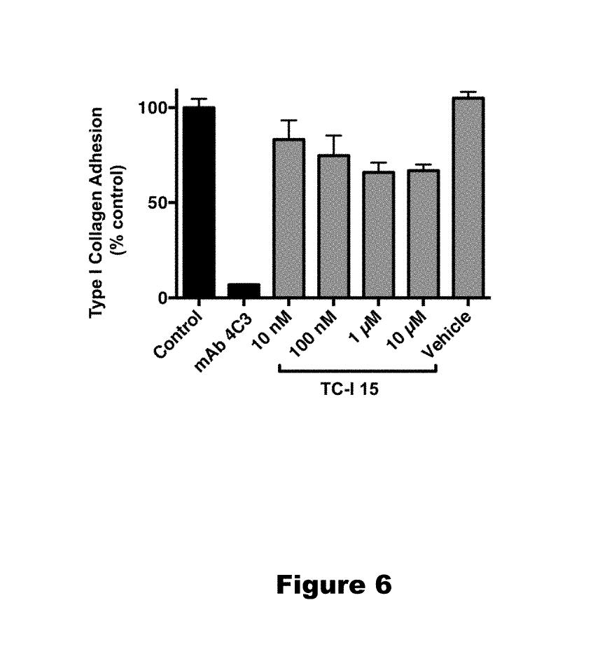

Having used the outlined strategy to identify function-blocking mouse antibodies, these reagents could be leveraged to generate humanized mAbs (14). From a therapeutic perspective, the broad distribution of the α₂β₁ integrin in normal tissues as well as its ability to ligate other ECM proteins [e.g., type IV collagen, laminin and type XXIII collagen (30, 57)] might raise concerns regarding potential toxicities associated with targeting strategies. However, it is noteworthy that α2-null mice are viable and fertile, and that α2-integrin-deficient human patients have been identified who present only with mild bleeding diatheses (58-61). Interestingly, small molecule α₂β₁ inhibitors have been developed as potential anti-thrombotics (62, 63), but preliminary studies indicate that these agents are not as effective as mAb 4C3, at least in terms of interfering with MDA-MB-231-type I collagen adhesive interactions (FIG. 6). Hence, it remains possible that mAb 4C3 exerts unique effects on carcinoma cell function that may not be recapitulated by small molecule inhibitors or α₂ integrin silencing. Finally, though the presented findings emphasize potential roles for α₂β₁ in neoplastic states, the integrin has also been implicated in fibrosis, inflammation, platelet-mediated thrombosis and angiogenesis, reinforcing the fact that similar targeting strategies can be applied in other disease states (64-70).

The experimental approach outlined herein allows for the rapid identification of new target antigens in an unbiased fashion as well as the isolation of murine monoclonal antibodies suitable for humanization. Though a human breast carcinoma cell line has been used as a proof-of-concept model, the approach is similarly amenable to the use of primary carcinoma cells or cancer stem cells. Indeed, primary human glioblastoma cancer stem cells have also been used to generate mAb libraries that have also been found to exert inhibitory effects with target identification in process (Table I). As such, the phenotypic screening stratagem, using either human cancer cell lines, primary cancer cells or even cancer cell-stromal cell composites (71), as well as more complex ECM-supplemented hydrogels to more accurately recapitulate the anticipated changes that occur in connective tissue composition during tumor progression (12, 72), will allow for the identification new targets and therapeutics in neoplastic as well as other disease states (e.g., fibrosis, acute/chronic inflammation, hypervascularization). The cell culture conditions may also be manipulated, for example, by application of hypoxic conditions.

### EXAMPLE 1

### Methods

MDA-MB-231 cells (ATCC) were embedded in mouse type I collagen hydrogels (1, 21) and the cell-matrix composite inoculated into 6 week-old Balb/c female mice for immunization. MDA-MB-231-reactive mAbs were isolated and screened for anti-proliferative activity in three-dimensional collagen constructs (1, 21).

Immunogen Preparation and Immunization

Type I collagen was isolated from mouse tail tendons as described (1, 21) and dissolved in 0.2% acetic acid at a final concentration of 2.7 mg/ml. Prior to gelation, the collagen solution was mixed with 10× MEM and 0.34 N NaOH at a ratio of 8:1:1 at 4° C. with MDA-MB-231 cells (1-5×10⁶) suspended in lml of this mixture. The carcinoma cell-collagen mixtures were incubated for 1 hour at 37° C. to allow for gelation and culture media (MEM supplemented with 10% FCS) added atop the gel. Collagen gel rigidity was assessed in a RFSII rheometer (Rheometrics) using dynamic shear mode, parallel plate geometry and a hydrated chamber as described (2). After a 4-day incubation period, the MDA-MB-231/collagen composites were washed extensively and recovered intact from 12-well plates or, alternatively, after the MDA-MB-231 cells were harvested from the gels by dissolving the collagen hydrogels with collagenase type 3 (Advance BioFactures Corp.). MDA-MB-231/collagen composites or isolated MDA-MB-231 cells were inoculated intraperitoneally into 6 week-old Balb/c female mice, followed by boosts at two-three week intervals for 3 months. Spleens were then removed and somatic cell hybridization performed with P3X63-Ag8.653 mouse myeloma cells as the fusion partner (3).

### Whole-Cell ELISA

Supernatants from hybridoma clones were assayed in a whole-cell ELISA format. MDA-MB-231 cells (1×10⁵) were added to 96-well V-bottom PVC plates (Corning) and cell pellets incubated for 1 hour at 4° C. with 50 μl of media supernatant from individual hybridoma cultures. After washing, MDA-MB-231 cells were then re-suspended in PBS with a horseradish peroxidase (HRP)-conjugated secondary antibody directed against mouse immunoglobulins (Pierce) for 1 hour at 4° C. Cells were then washed three times with PBS and HRP activity detected with a TMB substrate (Thermo Scientific).

Hybridomas giving rise to anti-MDA-MB-231-reactive mAbs were sub-cloned by limiting dilution and re-assayed for activity to ensure the isolation of monoclonal populations. Positive hybridomas were then used to generate ascites fluid by injection into mouse peritoneal cavities. The resulting ascites fluid was cleared of debris by centrifugation and antibodies purified by either Melon Gel Purification Resin (Thermo Scientific) or Protein G Resin (Thermo Scientific). Monoclonal antibody (mAb) isotype was determined by Rapid ELISA mouse mAb Isotyping Kit (Pierce). A control IgG1 mAb (3H5) was raised against dengue virus antigen (4). Following intraperitoneal injection, ascites fluid generated from the hybridoma cell line (ATCC) was purified by Protein G affinity chromatography. Both the control mAb 3H5 and the mAb 4C3 preparations were endotoxin-depleted by DeToxi-Gel column chromatography (Pierce) prior to use.

Cell Proliferation and Apoptosis Assays

For screening mAb anti-proliferative activity, MDA-MB-231 cells were embedded in type I collagen (10⁵ cells in a final type I collagen concentration of 2.2 mg/ml) or Matrigel (5 mg/ml) in the absence or presence of mAb 4C3 at the indicated concentrations and plated in 24-well plates in MEM/10% FCS. In selected experiments, the ability of mAb 4C3 to affect proliferative responses of human squamous cell carcinoma (74B), ovarian carcinoma (ES2) or fibrosarcoma (HT1080) cells (all obtained from ATCC) was assessed. Cell number was quantified by hemocytometry or using a Cell-Titer Glo kit (Promega). Caspases 3 and 7 activities were evaluated with a Caspase-Glo 3/7 kit (Promega).

### Affymetrix Expression Profiling and Analysis

Total mRNA was collected and purified using RNeasy Mini Kits (QIAGEN) (5). Sample quality was confirmed using a Bioanalyzer 2100 and all samples profiled on Affymetrix Mouse MG-430 PM expression array strips. Expression values for each probe set were calculated using the robust multi-array average (RMA) system (5) and filtered for genes with a fold change greater than 2-fold. Heatmaps of selected gene lists were generated using Gene Cluster 3.0 and TreeView 1.6 (5). Gene ontology analysis was performed using MetaCore from Thomson Reuters (version 6.11, build 41105).

### Chick Xenograft

RFP-transduced MDA-MB-231 were injected with a control IgG or 4C3 into the allantoic vein of 11-day-old, immune-incompetent chick embryos (6, 22). After a 6-day incubation period, vessel lumens were visualized by injecting chicks with GFP-labeled isolectin-B4. Confocal imaging of second harmonic-generated signals was used to analyze collagen fiber microstructure as described (7, 24). After an additional 1-hour incubation time, embryos were harvested, whole-mount tissue preparations taken distally from the injection site, and carcinoma cells identified by florescent microscopy. For quantification, MDA-MB-231 cells expressing firefly luciferase were injected in an identical fashion with control mAb 3H5 or mAb 4C3 in tandem with the carcinoma cells or 24 hours after the carcinoma cell innoculation. For imaging, eggs were injected i.v. with 100 μl luciferin (40 mg/ml in PBS) 10 minutes prior to removal of the lower chioroallantoic membrane. Membranes were washed with PBS and imaged for bioluminescence with a Xenogen IVIS 200.

### Mouse Xenograft Model

Luciferase-labeled MDA-MB-231 cells (1×10⁵) were injected via the intracardiac route with either 10 mg/kg of mAb 4C3 or a control IgG1 twice-weekly for 4 weeks and tumor progression by whole-body bioluminescent imaging as described (8). In selected experiments, cells were alternatively injected orthotopically in the 4th mammary gland with or without mAb 4C3. MicroCT analysis of bone lesions were imaged at 18-μm isotropic voxel resolution using Explore Locus SP (GE Healthcare Pre-Clinical Imaging) and calibrated three-dimensional images reconstructed (7, 24).

### Immunoaffinity Purification and Mass Spectrometry (MS) of Target Antigen

To identify the mAb 4C3 ligand, RIPA lysates of MDA-MB-231 cells (1 mg/ml) were pre-cleared with 5 μg control mouse IgG1 and Protein A/G beads (Santa Cruz). Monoclonal antibody mAb 4C3 (5 μg) was then incubated with Protein A/G beads overnight at 4° C. The beads were pelleted and washed with RIPA buffer, attached proteins solubilized in Laemmli sample buffer, and resolved on 10% SDS-PAGE gels (Bio-Rad). The immunoprecipitated protein was visualized by silver staining (Pierce), and the band was excised and subjected to in-gel digestion with porcine trypsin. Gel digests were analyzed by LC/MS/MS on a ThermoFisher LTQ Orbitrap XL mass spectrometer. Peptide ion data were searched and identified using Mascot and Scaffold at the University of Michigan Protein Structure Facility. To verify the identified ligand, immunoprecipitated protein was resolved on a SDS-PAGE gel, transferred to nitrocellulose membrane, and immunoblotted with a second antibody directed against human integrin α2 (Santa Cruz Biotechnology, sc-74466).

Tissue Histochemistry

Formalin-fixed, paraffin-embedded tissue blocks from de-identified patient samples (IRB protocol HUM000503390) were sectioned (5 μm) and placed on charged slides. Slides were deparaffinized in xylene and rehydrated through graded alcohols. Heat-Induced Epitope Retrieval (HIER) was performed in the Decloaking Chamber (Biocare Medical) with Target Retrieval at pH 6.0 (DakoCytomation). Slides were incubated in Peroxidazed (Biocare Medical) for 5 minutes to quench endogenous peroxidases and then incubated for 1.5 hours at 25° C. with rabbit monoclonal anti-α2 integrin (CD49b; Abcam LTD/Epitomics) diluted 1:200 (this monoclonal antibody produces staining superior to mAb 4C3). Antibody was detected with anti-rabbit Envision⁺ HRP Labelled Polymer (DakoCytomation) for 30 minutes at 25° C. HRP staining was visualized with the DAB⁺ Kit (DakoCytomation). Slides were counterstained in hematoxylin, blued in running tap water, dehydrated through graded alcohols, cleared in xylene and then mounted with Permount.

### Statistical Analysis

All results are presented as the mean±SEM of 3 or more experiments as indicated in the text. Significance was determined using the Student's t-test.

### EXAMPLE 2

### Characterization of Function-Blocking Monoclonal Antibodies Directed Against MDA-MB-231 Carcinoma Cells

MDA-MB-231 cells are a well-characterized, triple-negative breast carcinoma cell line whose gene expression profile closely recapitulates that found in human breast cancer tissues (17-19). Further, in a manner similar to human carcinomas expanding in vivo, the cell line undergoes rapid proliferative and tissue-invasive responses when cultured within three-dimensional type I collagen hydrogels in vitro (16, 20, 21). As such, MDA-MB-231 cells were embedded in covalently cross-linked networks of mouse type I collagen with an elastic modulus similar to that found in normal breast tissue [about 150 Pa (11)]. After a 3-day culture period, the human carcinoma cell-mouse matrix composite was then recovered and used as an immunogen to generate a panel of approximately 2500 mAbs (FIG. 1). To identify MDA-MB-231-reactive clones, whole cell-based ELISAs were then performed with about 350 of the mAbs scoring positive in initial screens. Each of the reactive clones was then expanded and the individual mAbs tested for their ability to inhibit the proliferative responses of MDA-MB-231 cells in three-dimensional culture (FIG. 1).

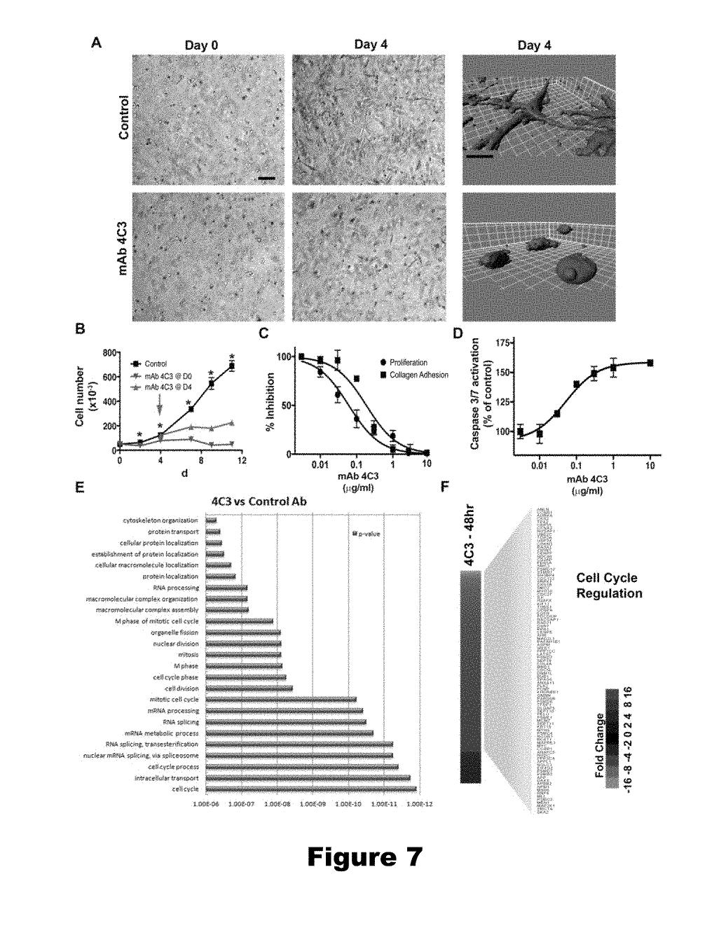

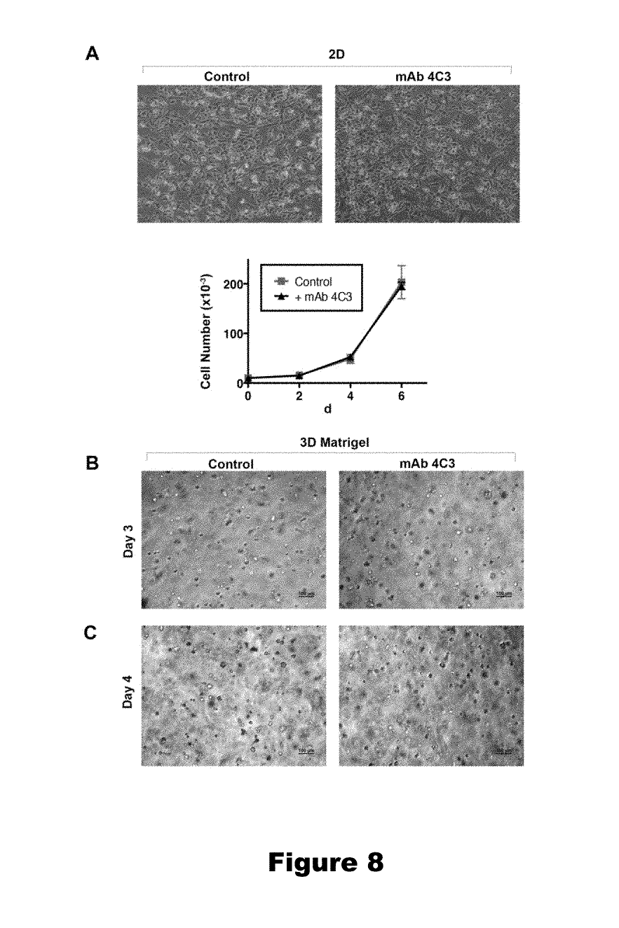

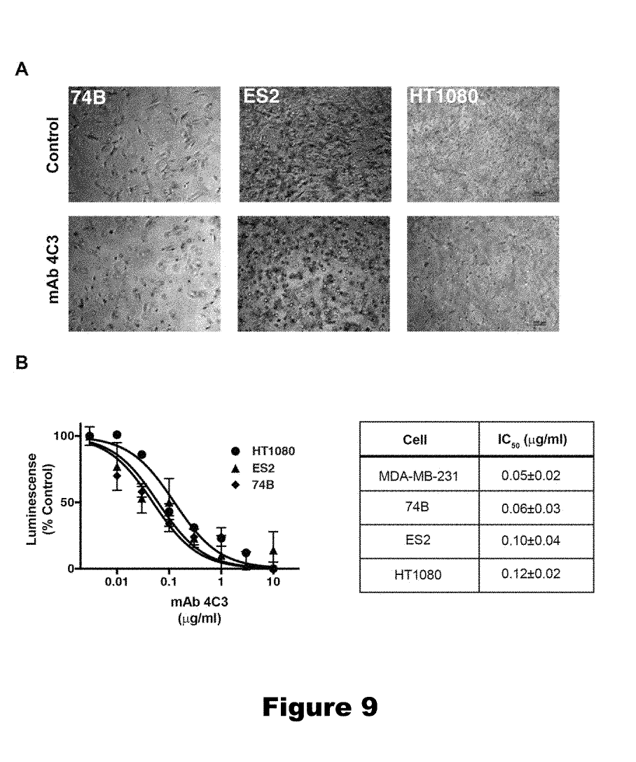

When embedded in three-dimensional type I collagen hydrogels, MDA-MB-231 cells rapidly alter their morphology from a spherical to bipolar, mesenchymal cell-like phenotype over the first 48 hours prior to the initiation of proliferative responses (FIG. 7A,B). Among the 15 mAbs displaying inhibitory activity in our initial screens, clone 4C3 was one of the more potent IgG1-class antibodies identified, displaying an ability to almost completely block MDA-MB-231 cell shape changes and proliferation in three-dimensional collagen (FIG. 7A,B). Moreover, inhibitory activity was not limited to a “preventative” protocol wherein mAb 4C3 was added at the start of the three-dimensional culture period; addition of the inhibitory mAb 4 days after the initiation of the culture period similarly inhibits carcinoma cell proliferation with an IC50 of approximately 0.5 μg/ml (FIG. 7B,C). Furthermore, 4C3 not only blocks MDA-MB-231 proliferative responses, but also initiates apoptosis in three-dimensional culture as assessed by caspase 3 and 7 activation (FIG. 7D). By contrast, when cultured under standard two-dimensional conditions atop tissue culture-treated plastic substrata or within three-dimensional Matrigel, an ECM extract that neither recapitulates the structure of normal basement membranes structure nor that of the interstitial matrix (1, 2), mAb 4C3 exerts no inhibitory effects on MDA-MB-231 cell function (FIG. 8). Interestingly, the anti-proliferative activity of mAb 4C3 is not confined to MDA-MB-231 carcinoma cells as similar inhibitory effects are observed with human squamous cell carcinoma, ovarian carcinoma and fibrosarcoma cell lines in three-dimensional culture (FIG. 9).

### EXAMPLE 3

Monoclonal Antibody 4C3 Exerts Global Effects on the MDA-MB-231 Transcriptome

In an effort to identify the potential signaling networks impacted by mAb 4C3, MDA-MB-231 cells were next cultured in three-dimensional type I collagen hydrogels in the presence of either a control IgG1 or mAb 4C3 for 48 hours (i.e., prior to the initiation of proliferative responses) and RNA harvested for gene expression profiling. Under these conditions, mAb 4C3 exerted global effects on gene expression with almost 1200 unique transcripts affected (i.e., 172 up-regulated and 1004 down-regulated transcripts, respectively, using a 2.0-fold cutoff). Consistent with its effect on MDA-MB-231 proliferation, GO analysis revealed that mAb 4C3 treatment elicits major alterations in cell cycle, regulation, RNA processing and cell division-related programs (FIG. 7E). Taken together, these results identify mAb 4C3 as a potent regulator of MDA-MB-231 cell function within the confines of a type I collagen-rich ECM.

### EXAMPLE 4

### Monoclonal Antibody 4C3 Prevents Post-Extravasation Carcinoma Growth In Vivo

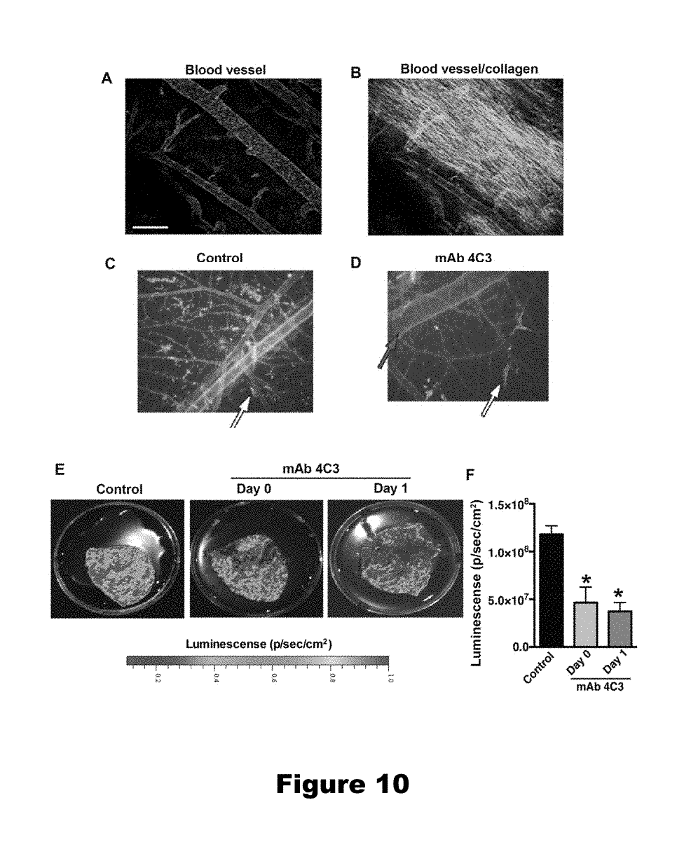

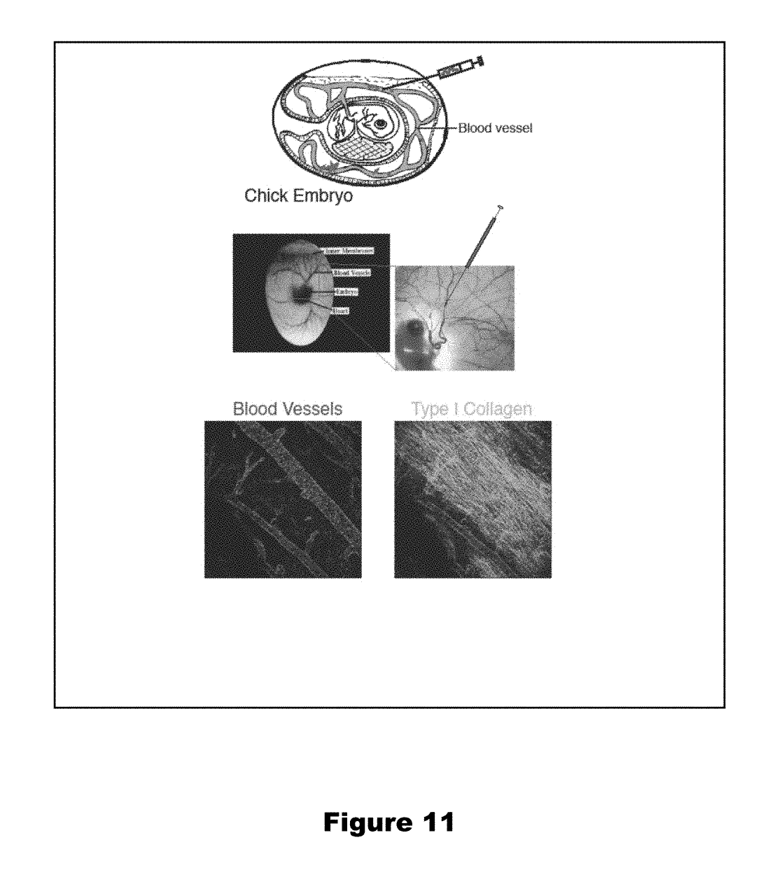

In our in vitro model, embedded carcinoma cells are individually surrounded by a network of type I collagen fibrils, a scenario similar to that encountered when circulating tumor cells extravasate from vascular or lymphatic beds and enmesh themselves within the perivascular interstitial matrix (1-3, 6, 12). To examine the inhibitory potential of mAb 4C3 in a post-extravasation program directly, we utilized a live embryonic chick xenograft model that faithfully recapitulates carcinoma cell behavior (e.g., proliferation) in mouse xenograft models (22, 23). As shown in FIGS. 5A and 17, the chick chorioallantoic membrane vasculature is readily visualized by confocal laser microscopy. Further, using second harmonic generation to image type I collagen fibrils in situ (24), blood vessels are shown to be uniformly invested by a dense collagenous network (FIGS. 5B and 17). As such, fluorescently-tagged MDA-MB-231 cells were injected into the host vasculature of 11-day-old, immuno-incompetent chick embryos in tandem with a control IgG1 or mAb 4C3, and post-extravasation growth monitored. Following a 6-day culture period in vivo, extravasated MDA-MB-231 cells initiate proliferative activity in close association with the chick vasculature (FIG. 10C). As can be seen in the bottom two panels of FIG. 11, blood vessels (colored green) within chick tissues are surrounded by a dense layer of type I collagen, visualized by second harmonic generation microscopy in FIG. 11. By contrast, in the presence of mAb 4C3, MDA-MB-231 cell proliferation is inhibited markedly wherein tumor colony formation is readily monitored by both visual inspection and quantification of luminescent signals using luciferase-tagged carcinoma cells (FIG. 10C,D). To rule out the possibility that 4C3 blocks proliferative responses by interfering with MDA-MB-231 extravasation itself, carcinoma cells were injected into the chick vasculature, and after a 24-hour period in which extravasation is complete (23), mAb 4C3 was introduced intravascularly. Even under these conditions, mAb 4C3 exerts potent inhibitory effects equivalent to those obtained when the antibody is introduced at the start of the in vivo assay (FIG. 10C,D).

### EXAMPLE 5

### Anti-Metastatic Activity of Monoclonal Antibody 4C3 in a Mouse Xenograft Model

Unlike humans, where mammary tissues are dominated by type I collagen, the mouse mammary gland contains only small amounts of type I collagen that is largely confined to periductal regions alone, thus rendering mouse xenograft orthotopic models less useful for analyzing carcinoma cell-type I collagen matrix interactions (25). Alternatively, the organic matrix of mouse bone—like that of humans—is largely comprised of type I collagen (17, 26-28).

Further, bone is a frequent site of breast cancer metastatic activity in human disease (17). As such, following intracardiac injection, the ability of luciferase-tagged MDA-MB-231 to generate bone metastatic lesions was assessed in nude mouse recipients in the presence of control IgG1 or mAb 4C3 by in vivo imaging as well as microCT analyses over a 28-day assay period. Mice were treated for four weeks with twice-weekly dosages of 10 mg/kg of the control mAb, MDA-MB-231 cells generated large tumors in the mandible, hindlimb and spine of the inoculated mice, as revealed by luminescent imaging (FIG. 3A,B). By contrast, in the mAb 4C3-treated group, carcinoma growth in the mandible and hindlimb is impaired with significant inhibitory effects recorded in vertebral metastases where bone-erosive lesions were readily observed in microCT scans of the control antibody-treated group (FIG. 3A-C). Whereas approximately 50% of the control antibody-treated mice required euthanization due to spinal cord compression and resulting limb paralysis, fewer than 20% of the mAb 4C3-treated mice were similarly affected, consistent with the ability of mAb 4C3 to block the progression of bony metastases (FIG. 3D).

These results are most consistent with either the ability of mAb 4C3 to exert direct bone-sparing effects or the inability of mAb 4C3-treated tumor cells to proliferate within the vertebral compartment. Indeed, whereas the femur marrow compartment is large, allowing unrestricted cancer cell growth independent of direct tumor-matrix interactions, MDA-MB-231 proliferation was potently suppressed within the space-restricted mandibular and spinal compartments (FIG. 3B). Hence, it has been demonstrated that monoclonal antibody 4C3 blocks tumor expansion in collagen-rich environments in vitro and in vivo while displaying inhibitory effects on bony metastases and their sequelae. Finally, though efforts to date have focused on the impact of mAb 4C3 on breast carcinoma behavior, it should be stressed that virtually all carcinoma cell types express α2β1 following their invasion into surrounding tissues (e.g., ovarian, pancreatic, prostate, colon), supporting the expectation of a more global role for monoclonal antibody 4C3 as a cancer therapeutic (39, 46, 73, 74, 75). Indeed, studies have indicated that the proliferative responses of cultured human squamous cell carcinoma, human ovarian carcinoma and human fibrosarcoma are also inhibited by the 4C3 monoclonal antibody.

### EXAMPLE 6

### Identification of the Monoclonal Antibody 4C3 Target Antigen and its Expression in Human Breast Cancer Bone Metastases

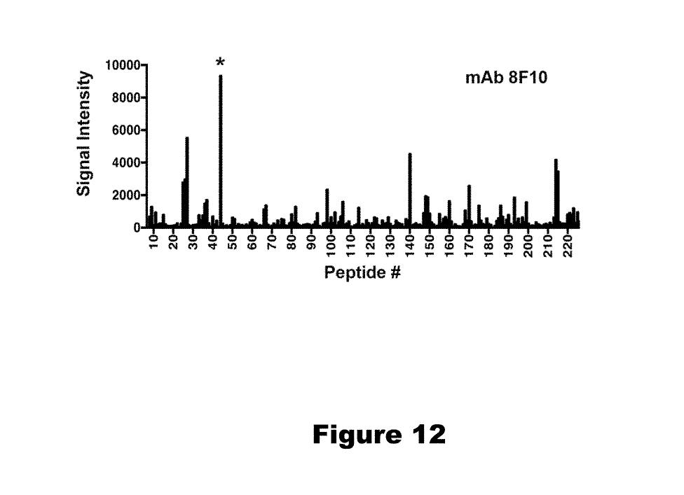

To next identify the target antigen recognized by mAb 4C3, whole cell lysates of MDA-MB-231 cells were applied to immuno-affinity columns constructed using the purified antibody as the capturing agent. Following antigen recovery, a major bond of about 150 kD was isolated and submitted for mass spectrometric analysis following trypsin fragmentation (FIG. 4A). Bio-informatic analysis of the generated fragments identified the target antigen as the integrin subunit, alpha 2 (α₂) (29, 30). Immunoprecipitation of MDA-MB-231 lysates with mAb 4C3, followed by immunoblotting with an independent anti-α₂ antibody further confirmed the target antigen as the α₂ integrin subunit (FIG. 4B). Consistent with the fact that α₂ integrin subunit only forms heterodimeric complexes with the β1 integrin to generate the dominant mammalian type I collagen receptor, α₂β₁ peptide mapping of mAb 4C3 interactions with the α₂ subunit identified a major epitope that lies within the α-I domain of the integrin, the dominant type I collagen recognition site of the α₂β₁ heterodimer (29, 30) (FIG. 4C). As normal cell trafficking is minimal in adult tissues (except for myeloid cells that do not express α2β1), and as all cancer cells must traffic through—and grow within—type I collagen-rich tissues (2), mAb 4C3 is expected to possess qualities that allow it to serve as a broad-acting cancer therapeutic. Interestingly, human patients that carry mutations in the α2 integrin that prevents its normal expression are only mildly affected with marginal increases in bleeding tendencies due to the fact that platelets express low levels of the α2 integrin (58). In addition to mAb 4C3, a second, inhibitory α₂ integrin-reactive mAb that was identified independently in our screen (mAb 8F10) also bound to a distinct, but overlapping, epitope located within the α-I domain (FIG. 12). As expected from its collagen-binding properties, mAb 4C3 inhibits MDA-MB-231 adhesive interactions with type I collagen (FIG. 7C).

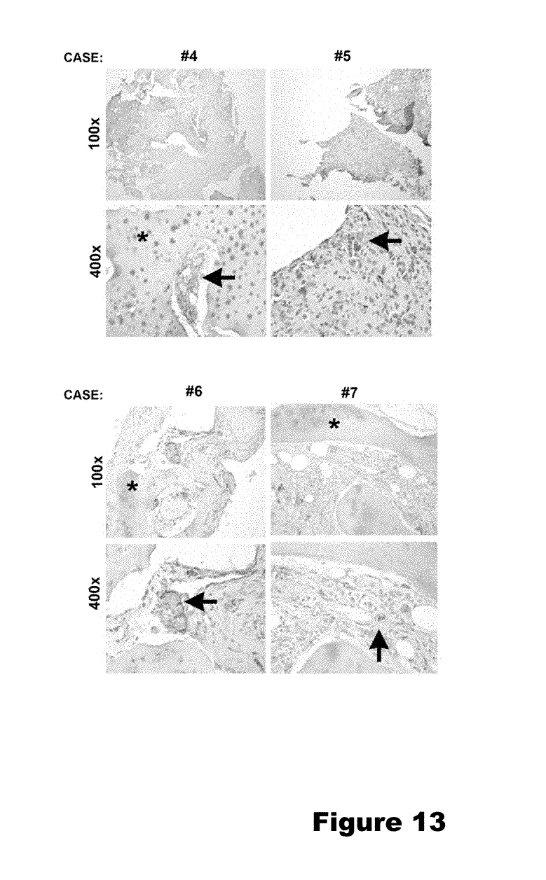

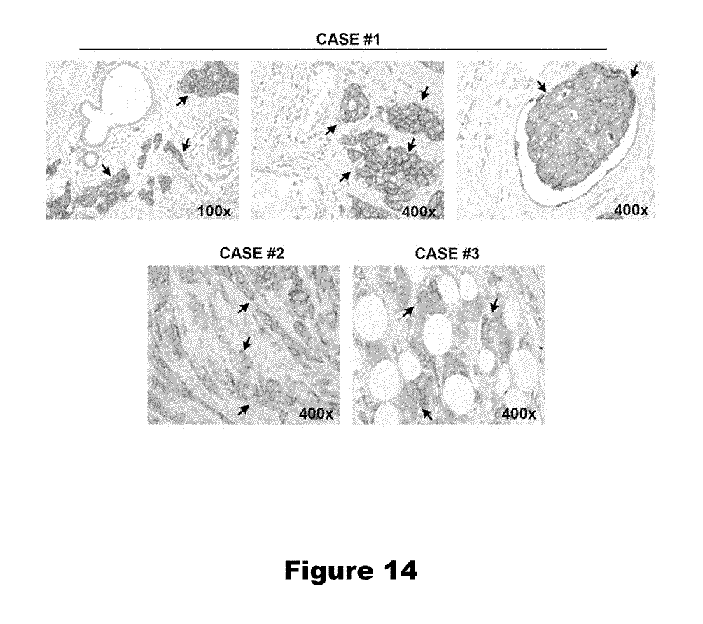

Given these results, and earlier studies demonstrating the ability of MDA-MB-231 cells to form α₂β₁-dependent adhesive interactions with bone matrices in vitro (26-28), we sought to determine whether our in vitro model accurately predicts α₂ integrin expression patterns found in type I collagen-rich metastatic lesions recovered from human breast cancer patients. As such, bone biopsies were obtained from a series of 7 patients with metastatic disease and immuno-stained for α₂ expression. Validating the results of our in vitro and xenograft models, all 7 patients expressed α₂ in breast cancer cells in bone metastatic sites with both carcinoma cells as well as surrounding vascular endothelial cells scoring positive in blinded analyses (FIG. 5 and FIG. 13). As archived biopsy material was available from the original primary breast cancer site in a subset of three of these patients, and type I collagen levels in human breast tissue is distinctly higher than that found in the mouse mammary gland (25), α₂β₁ staining was assessed in these samples as well. Interestingly, distinct α₂ integrin expression is detected in breast carcinoma cells in each of these patients (with weaker staining localized to normal myoepithelial cells), including tumor microemboli found within lymphatic vessels (FIG. 14).

### EXAMPLE 7

### Subtractive Immunization

Having generated a panel of monoclonal antibodies against human breast carcinoma cells, functional screening of the panel was initiated to demonstrate that i) inhibitory antibodies can be elicited, ii) target antigens identified, and iii) functional activity assigned in vivo. To facilitate the identification of inhibitory antibodies, a subtractive immunization technique was employed that enriched for antibodies specifically binding to tumor-specific antigens.

The subtractive immunization procedure involves immunizing mice with the normal cellular counterpart of the human carcinoma (e.g., in the case of breast cancer, animals are primed with normal human mammary epithelial cells) and then treated with the immunosuppressive agent, cyclophosphamide. These mice are then prevented from maintaining an immune response against antigens found on the normal human epithelial cells, a process resulting in tolerized mice. The tolerized mice are then challenged by injection of human carcinoma cells. This experimental protocol results in an enhanced immune response directed toward antigens found specifically on the tumor cells. The versatility of using subtractive immunization to enrich for antibodies of interest is apparent in the realization that initial exposure to a control counterpart can be used to reduce the presence of antibodies not specifically binding to a target of interest upon elicitation of antibodies to a three-dimensional immunogen containing the target of interest associated with a cell exhibiting a disease, disorder or condition, or associated with an extracellular compound such as a protein, or simply associated with the hydrogel of type I collagen, fibrin, or both. Exemplary control counterparts include a healthy, or normal, counterpart in the form of a healthy cell of the same type as a diseased cell, or the extracellular microenvironment from a healthy organism that corresponds to the extracellular microenvironment containing a target for a disease, disorder or condition of interest.

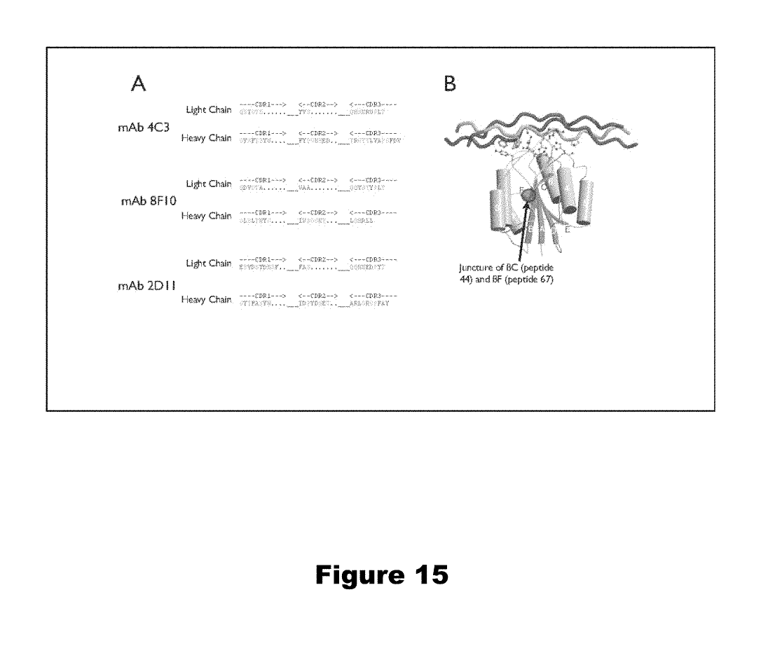

In addition to mAb 4C3, two other monoclonal antibodies were identified in our screens that recognize the α2 integrin subunit, monoclonal antibodies 8F10 and 2D11. To characterize the antibody-antigen binding site, the CDR domains for each have been identified (FIG. 15A; see also, FIG. 12). Further, we have undertaken epitope-mapping and have found two peptides recognized by each of these antibodies that are found in the α-I domain of the α2 integrin subunit (FIG. 15B). Interestingly, the a-I domain is responsible for binding type I collagen, providing a molecular rationale for the biological activity of these antibodies (78).

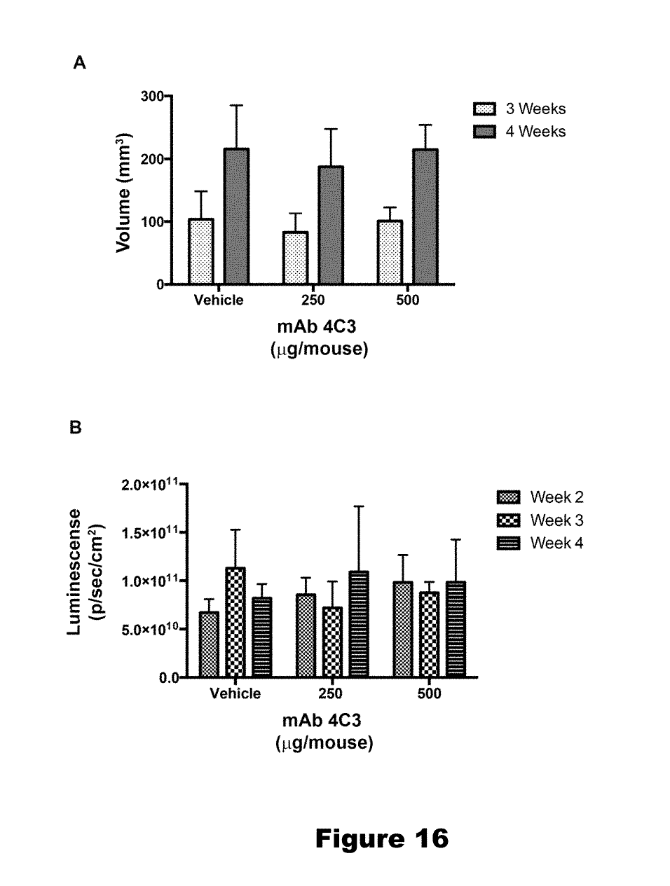

As monoclonal antibody 4C3 was raised against a human breast carcinoma cell line, studies were performed to determine the ability of the antibody to recognize normal and cancerous breast tissue by immunohistochemistry. As such, a human breast tumor tissue array was stained with mAb 4C3 and counterstained with hematoxylin. As shown in FIGS. 9 and 16, mAb 4C3 lightly stained epithelial cells in normal human mammary ducts, as well as portions of the surrounding stromal tissue. In contrast, several cancer types, including examples of ductal carcinoma, displayed markedly enhanced staining with mAb 4C3. Staining of tumor tissue with mAb 4C3 is consistently enhanced relative to normal tissue. See also, FIGS. 10 and 11.

Following cancer cell inoculation, tumor cells (colored orange) extravasate from the chick vasculature, invade into the surrounding, type I collagen-rich extracellular environment and form nascent tumors during a 6-day culture period (FIG. 10). As such, this model provides a convenient means to study cancer cell invasion and proliferative potential in vivo, as well as providing a rapid approach for evaluating the ability of potential therapeutics to inhibit these critical processes. Importantly, mAb 4C3 markedly inhibits the ability of MDA-MB-231 cells to maintain proliferative activity within the surrounding ECM (FIG. 10). Similar, if not identical results, are obtained when mAb 4C3 treatment is delayed for 24 hours after cancer cell inoculation to allow extravasation to proceed to completion. Thus, mAb 4C3 exerts potent anti-proliferative activity in vivo.

To further explore activity in vivo, a mouse bone metastasis model was used wherein human breast cancer MDA-MB-231 cells were injected into the left cardiac ventricle. Cells introduced in this manner tend to form metastases in the hindlimb and mandible (76, 77). Following confirmation of successful intracardiac delivery, mice were treated with twice-weekly dosages of 10 mg/kg mAb 4C3 for 4 weeks and tumor progression monitored by luminescent imaging (FIG. 3A). Treatment with mAb 4C3 inhibited hindlimb and mandible tumor progression with significant effects on the tumor growth localized to the spinal region (FIG. 3B). Further, at the end of the treatment period, it was necessary to euthanize about 50% of the control animals due to hindlimb paralysis, a common manifestation of spinal nerve damage secondary to vertebral collapse (FIG. 3C) (76, 77). By contrast, less than 20% of the 4C3-treated mice displayed paralysis during these experiments (FIG. 3D). These results are most consistent with either the ability of mAb 4C3 to exert direct bone-sparing effects or the inability of mAb 4C3-treated tumor cells to proliferate within the vertebral compartment. Indeed, whereas the femur marrow compartment is large, allowing unrestricted cancer cell growth independent of direct tumor-matrix interactions, MDA-MB-231 proliferation was potently suppressed within the space-restricted mandibular and spinal compartments (FIG. 3B). Hence, it has been demonstrated that monoclonal antibody 4C3 blocks tumor expansion in collagen-rich environments in vitro and in vivo while displaying inhibitory effects on bony metastases and their sequelae. Finally, though efforts to date have focused on the impact of mAb 4C3 on breast carcinoma behavior, it should be stressed that virtually all carcinoma cell types express α2β1 following their invasion into surrounding tissues (e.g., ovarian, pancreatic, prostate, colon), supporting the expectation of a more global role for monoclonal antibody 4C3 as a cancer therapeutic (79-83). Indeed, studies have indicated that the proliferative responses of cultured human squamous cell carcinoma, human ovarian carcinoma and human fibrosarcoma are also inhibited by the 4C3 monoclonal antibody.

A technology platform has been validated that allows for the rapid identification of anti-human cancer-neutralizing monoclonal antibodies. Isolated murine antibodies can be used as templates for the generation of humanized monoclonals for therapeutic intervention while identified target antigen may be leveraged to direct the synthesis of small-molecule inhibitors. Further, the disclosed technology is not only amenable to the use of well-characterized cancer cell lines, but also primary cancer cells, as well as cancer stem cells. Finally, in addition to its use in identifying a viable ligand target, mAb 4C3 has substantial activity in vivo, indicating its capacity as a therapeutic entity in its own right.

### EXAMPLE 8

### General Applicability of the Disclosed Technology

Following the successful elicitation of mAb 4C3 specifically recognizing the α2 integrin subunit as preferentially presented on cancer cells, additional experiments were performed to demonstrate the versatility of the technology. Using each of the MDA-MB-231 and SUM159 breast carcinoma cell lines, three-dimensional hydrogels composed of one or the other cancer cell lines embedded in a type I collagen matrix were used as immunogens in mice. The immunization schedules for some of these experiments involved the subtractive immunization approach described in Example 7 and outlined in FIG. 2. The results of these experiments reveal that 111 ELISA-positive antibody clones were obtained that specifically recognized and bound to the MDA-MB-231 cancer cell line cells and 76 ELISA-positive antibody clones specifically recognized and bound to the SUM159 stem cell cancer line cells. Thus, the data disclosed herein show that the technology elicited multiple specific antibodies to biomolecular target molecules on two different breast cancer cell types. In addition, 62 ELISA-positive antibody clones were obtained that specifically recognized and bound to biomolecular target molecules on Glioblastoma cells and 50 different ELISA-positive antibody clones specifically recognized and bound to ovarian carcinoma cells. Experiments underway lead to the expectation that multiple independent ELISA-positive antibody clones will be obtained that specifically recognize and bind to biomolecular target molecules on either pancreatic carcinoma cells or melanoma cells, consistent with the expectation that the technology is broadly applicable to biomolecular target molecules on any cancer cell line and, indeed, any cell exhibiting a disease, disorder or condition.

Additional data is presented in Table 1, with those immunogens delivered using subtractive immunization indicated by including “Subtrn” in the immunogen name. Column 1 of Table 1 provides the name of the elicited monoclonal antibody, column 2 identifies the isotype of that antibody (heavy and light chains), column 3 discloses the cancer cell-derived immunogen that elicited the antibody, column 4 identifies the immunoprecipitate as a means of identifying the target of the antibody and column 5 reveals whether a signal was obtained from a lysate of the relevant cancer cell line using the indicated monoclonal antibody. Apparent from Table 1 is the fact that the disclosed technology is capable of eliciting monoclonal antibodies that recognize a variety of targets on cancer cell lines. Of note, each of the identified antibodies blocked tumor cell proliferation in the live chick xenograft model to a degree comparable to that observed with mAb 4C3. Further, these antibodies were generated by using (or not using) a subtractive immunization protocol. Still further, Table 1 shows that growth-inhibitory monoclonal antibodies were elicited to a series of distinct targets on each of the cancer cell lines used in the three-dimensional hydrogel immunogen (e.g., α2-integrin, α-enolase, calnexin, CD44, filamin, vimentin, and fibrinogen). Additionally, the data in Table 1 establish that a variety of antibody isotypes (e.g., IgG, IgM) and sub-isotypes (e.g., IgG1, IgG2) are amenable to elicitation using the technology disclosed herein.

In addition to establishing the advances noted in the above paragraph, the experiment yielding the data collected in Table 1 showed that each of the antibodies listed in the Table exhibited potent growth-inhibitory activity in the chick embryo model. Thus, the experimental results establish that the disclosed technology is effective in eliciting anti-target antibodies, and those anti-target antibodies have the functional effect of inhibiting the growth of the cell type used to elicit the antibody. Thus, without advance designation or even knowledge of a cellular target, the technology produced antibodies to cellular targets of interest, and those anti-target antibodies were functional in inhibiting the growth of the desired cell type.

Based on the disclosures herein, one of ordinary skill in the art would understand that the technology allows for the preparation of an immunogen in a three-dimensional environment that preserves or mimics the in vivo architecture, thereby maximizing the opportunity to obtain functionally useful (e.g., diagnostically, prophylactically and/or therapeutically useful, or useful in ameliorating a symptom of a disease, disorder or condition) antibodies to any number of immunogenic targets on a wide variety of cell types, including any type of cancer cell, fibrotic cell, or cell involved in either pathologic angiogenesis or inflammatory (pro-inflammatory) diseases, disorders or conditions. In considering fibrotic cells, there are not only the fibroblasts depositing the fibrous compositions, but the typically injured cells providing the signals ultimately leading to deposition of fibrous material by fibroblasts. Thus, markers for fibrotic disease include cell-surface markers associated with a variety of cells in addition to fibroblasts. For pathologic angiogenesis diseases, the disclosure comprehends a diseased endothelial cell, smooth muscle cell, pericyte or mesenchymal stem cell. For inflammatory diseases, contemplated are leukocytes, including any leukocyte type or sub-type. Moreover, for each of these aspects of the disclosure, human cells comprising human cells are contemplated.

TABLE 1
Active mAb Summary

| mAb | Isotype | Immunogen | I.P. | Western |
|---|---|---|---|---|
| 770.4C3 | IgG1, kappa | MDA-MB-231/collagen | Alpha2 Integrin | No signal |
| 774.8F10 | IgG1, kappa | MDA-MB-231/collagen | Alpha2 Integrin | No signal |
| 778.2D11 | IgG1, kappa | GBM | Alpha2 Integrin | No signal |
| 806.5C7 | IgM, kappa | 231-Subtrn | α-enolase | 35k band |
| 806.7G9 | IgM, kappa | 231-Subtrn | α-enolase | 35k band |
| 804.10G2 | IgM, kappa | SUM159-Subtrn | Calnexin | 80k band |
| 810.1C11 | IgG1, kappa | SUM159-Subtrn | Calnexin | 80k band |
| 806.2F5 | IgG2b, kappa | 231-Subtrn | CD44 | 75k band |
| 810.1C9 | IgG1, kappa | SUM159-Subtrn | Filamin | 250k band |
| 774.5G7 | IgM, kappa | MDA-MB-231/collagen | Vimentin | No signal |
| 784.2B4 | IgM, kappa | SUM159 | Vimentin | No signal |
| | | | HSPA8/HSPA5 | |
| | | | Fibrinogen | |
| 774.2F6 | IgM, kappa | MDA-MB-231/collagen | Did not I.P. | No signal |
| 804.8C9 | IgG1, kappa | SUM159-Subtrn | Did not I.P. | No signal |
| 806.7D2 | IgG3, kappa | 231-Subtrn | Did not I.P. | No signal |
| 785.2F4 | IgM, lambda | 231 ex vivo | Not assessed | No signal |

The experimental data disclosed herein establish that the disclosed methods for eliciting target-specific antibodies have wide applicability in that the disclosed methods engineer immunogens in three-dimensional forms that more closely resemble the in vivo microenvironment of a target. Antibodies elicited to such immunogens are reasonably expected to exhibit a higher degree of specific binding to the target molecule in vivo because the antibodies were elicited using forms of the target more closely matching the three-dimensional in vivo form of the target than immunogens known in the art. The potentially increased complexity of an initial polyclonal antibody response is offset by the realization that once an antibody specifically binding the target of interest is obtained, there is an increased likelihood that such an antibody will bind to the target in vivo, providing the intended beneficial effect on target activity. Moreover, the potentially increased complexity of an initial polyclonal antibody response can be reduced by incorporating the subtractive immunization procedure disclosed herein.

The foregoing description establishes that the disclosed technology has wide applicability in harnessing the immune response to diagnose, prevent, treat or ameliorate the symptom of a wide variety of diseases, disorders or conditions afflicting man, domesticated animals such as livestock or pets, and wild animals. This wide applicability to diagnostics, prophylactics, including vaccine development, therapeutics and amelioration of disease symptoms is a result of the broad applicability of the disclosed technology to immunological approaches to disease, disorder or condition diagnosis, prevention or treatment.

### REFERENCES

1. Rowe R G & Weiss S J (2008) Breaching the basement membrane: who, when and how? Trends Cell Biol 18:560-574.
2. Rowe R G & Weiss S J (2009) Navigating ECM barriers at the invasive front: the cancer cell-stroma interface. Annu Rev Cell Dev Biol 25:567-595.
3. Cheung K J, Gabrielson E, Werb Z, & Ewald A J (2013) Collective invasion in breast cancer requires a conserved basal epithelial program. Cell 155:1639-1651.
4. Lyons T R, et al. (2011) Postpartum mammary gland involution drives progression of ductal carcinoma in situ through collagen and COX-2. Nat Med 17:1109-1115.
5. Nguyen-Ngoc K V, et al. (2012) ECM microenvironment regulates collective migration and local dissemination in normal and malignant mammary epithelium. Proc Natl Acad Sci USA 109:E2595-2604.
6. Egeblad M, Rasch M G, & Weaver V M (2010) Dynamic interplay between the collagen scaffold and tumor evolution. Curr Opin Cell Biol 22:697-706.
7. Lu P, Weaver V M, & Werb Z (2012) The extracellular matrix: a dynamic niche in cancer progression. J Cell Biol 196:395-406.
8. Barkan D, et al. (2010) Metastatic growth from dormant cells induced by a col-I-enriched fibrotic environment. Cancer Res 70:5706-5716.
9. Cox T R, et al. (2013) LOX-mediated collagen crosslinking is responsible for fibrosis-enhanced metastasis. Cancer Res 73:1721-1732.
10. Salo S, et al. (2013) Human bone marrow mesenchymal stem cells induce collagen production and tongue cancer invasion. PLoS One 8:e77692.
11. Levental K R, et al. (2009) Matrix crosslinking forces tumor progression by enhancing integrin signaling. Cell 139:891-906.
12. Naba A, Clauser K R, Lamar J M, Carr S A, & Hynes R O (2014) Extracellular matrix signatures of human mammary carcinoma identify novel metastasis promoters. eLife 3:e01308.
13. Swinney D C & Anthony J (2011) How were new medicines discovered? Nat Rev Drug Discov 10:507-519.
14. Sliwkowski M X & Mellman I (2013) Antibody therapeutics in cancer. Science 341:1192-1198.
15. Rust S, et al. (2013) Combining phenotypic and proteomic approaches to identify membrane targets in a ‘triple negative’ breast cancer cell type. Mol Cancer 12:11.
16. Sabeh F, Shimizu-Hirota R, & Weiss S J (2009) Protease-dependent versus-independent cancer cell invasion programs: three-dimensional amoeboid movement revisited. J Cell Biol 185:11-19.
17. Kang Y, et al. (2003) A multigenic program mediating breast cancer metastasis to bone. Cancer Cell 3:537-549.
18. Bos P D, et al. (2009) Genes that mediate breast cancer metastasis to the brain. Nature 459:1005-1009.
19. Minn A J, et al. (2005) Genes that mediate breast cancer metastasis to lung. Nature 436:518-524.
20. Hotary K B, et al. (2003) Membrane type I matrix metalloproteinase usurps tumor growth control imposed by the three-dimensional extracellular matrix. Cell 114:33-45.
21. Sabeh F, et al. (2004) Tumor cell traffic through the extracellular matrix is controlled by the membrane-anchored collagenase MT1-MMP. J Cell Biol 167:769-781.
22. Ota I, Li X Y, Hu Y, & Weiss S J (2009) Induction of a MT1-MMP and MT2-MMP-dependent basement membrane transmigration program in cancer cells by Snail1. Proc Natl Acad Sci USA 106:20318-20323.
23. Koop S, et al. (1996) Independence of metastatic ability and extravasation: metastatic ras-transformed and control fibroblasts extravasate equally well. Proc Natl Acad Sci USA 93:11080-11084.
24. Tang Y, et al. (2013) MT1-MMP-dependent control of skeletal stem cell commitment via a beta1-integrin/YAP/TAZ signaling axis. Dev Cell 25:402-416.
25. Maller O, Martinson H, & Schedin P (2010) Extracellular matrix composition reveals complex and dynamic stromal-epithelial interactions in the mammary gland. J Mammary Gland Biol Neoplasia 15:301-318.
26. Ibaragi S, et al. (2011) Induction of MMP-13 expression in bone-metastasizing cancer cells by type I collagen through integrin alpha1beta1 and alpha2beta1-p38 MAPK signaling. Anticancer Res 31:1307-1313.
27. Lundstrom A, Holmbom J, Lindqvist C, & Nordstrom T (1998) The role of alpha2 beta1 and alpha3 beta1 integrin receptors in the initial anchoring of MDA-MB-231 human breast cancer cells to cortical bone matrix. Biochem Biophys Res Commun 250:735-740.
   28. van der P, et al. (1997) Attachment characteristics and involvement of integrins in adhesion of breast cancer cell lines to extracellular bone matrix components. Lab Invest 77:665-675.
29. Emsley J, Knight C G, Farndale R W, Barnes M J, & Liddington R C (2000) Structural basis of collagen recognition by integrin alpha2beta1. Cell 101:47-56.
30. Leitinger B (2011) Transmembrane collagen receptors. Annu Rev Cell Dev Biol 27:265-290.
31. Kurosawa G, et al. (2008) Comprehensive screening for antigens overexpressed on carcinomas via isolation of human mAbs that may be therapeutic. Proc Natl Acad Sci USA 105:7287-7292.
32. Kurosawa G, et al. (2011) Selection and analysis of anti-cancer antibodies for cancer therapy obtained from antibody phage library. Cancer Sci 102:175-181.
33. Harunaga J S & Yamada K M (2011) Cell-matrix adhesions in 3D. Matrix Biol 30:363-368.
34. Willis A L, Sabeh F, Li X Y, & Weiss S J (2013) Extracellular matrix determinants and the regulation of cancer cell invasion stratagems. J Microsc 251:250-260.
35. Gill B J, et al. (2012) A synthetic matrix with independently tunable biochemistry and mechanical properties to study epithelial morphogenesis and EMT in a lung adenocarcinoma model. Cancer Res 72:6013-6023.
36. Grzesiak J J, Vargas F, & Bouvet M (2010) Divalent cations modulate alpha2beta1 integrin-mediated malignancy in a novel 3-dimensional in vitro model of pancreatic cancer. Pancreas 39:904-912.
37. Hall C L, Dai J, van Golen K L, Keller E T, & Long M W (2006) Type I collagen receptor (alpha 2 beta 1) signaling promotes the growth of human prostate cancer cells within the bone. Cancer Res 66:8648-8654.
38. Hall C L, et al. (2008) Type I collagen receptor (alpha2beta1) signaling promotes prostate cancer invasion through RhoC GTPase. Neoplasia 10:797-803.
39. Kirkland S C (2009) Type I collagen inhibits differentiation and promotes a stem cell-like phenotype in human colorectal carcinoma cells. Br J Cancer 101:320-326.
40. Kirkland S C & Ying hours (2008) Alpha2beta1 integrin regulates lineage commitment in multipotent human colorectal cancer cells. J Biol Chem 283:27612-27619.
41. Kostenuik P J, Sanchez-Sweatman O, Orr F W, & Singh G (1996) Bone cell matrix promotes the adhesion of human prostatic carcinoma cells via the alpha 2 beta 1 integrin. Clin Exp Metastasis 14:19-26.
42. Li X, et al. (2013) Lung cancer cells that survive ionizing radiation show increased integrin alpha2beta1- and EGFR-dependent invasiveness. PLoS One 8:e70905.
43. Lochter A, Navre M, Werb Z, & Bissell M J (1999) alpha1 and alpha2 integrins mediate invasive activity of mouse mammary carcinoma cells through regulation of stromelysin-1 expression. Mol Biol Cell 10:271-282.
44. Shield K, et al. (2007) Alpha2beta1 integrin affects metastatic potential of ovarian carcinoma spheroids by supporting disaggregation and proteolysis. J Carcinog 6:11.
45. Valles A M, Boyer B, Tarone G, & Thiery J P (1996) Alpha 2 beta 1 integrin is required for the collagen and FGF-1 induced cell dispersion in a rat bladder carcinoma cell line. Cell Adhes Commun 4:187-199.
46. Van Slambrouck S, Jenkins A R, Romero A E, & Steelant W F (2009) Reorganization of the integrin alpha2 subunit controls cell adhesion and cancer cell invasion in prostate cancer. Int J Oncol 34:1717-1726.
47. Vuoriluoto K, Hognas G, Meller P, Lehti K, & Ivaska J (2011) Syndecan-1 and -4 differentially regulate oncogenic K-ras dependent cell invasion into collagen through alpha2beta1 integrin and MT1-MMP. Matrix Biol 30:207-217.
48. Yang C, et al. (2003) Integrin alpha1beta1 and alpha2beta1 are the key regulators of hepatocarcinoma cell invasion across the fibrotic matrix microenvironment. Cancer Res 63:8312-8317.
49. Ramirez N E, et al. (2011) The alpha(2)beta(1) integrin is a metastasis suppressor in mouse models and human cancer. J Clin Invest 121:226-237.
50. Chan B M, Matsuura N, Takada Y, Zetter B R, & Hemler M E (1991) In vitro and in vivo consequences of VLA-2 expression on rhabdomyosarcoma cells. Science 251:1600-1602.
51. Kato H, et al. (2012) The primacy of beta1 integrin activation in the metastatic cascade. PLoS One 7:e46576.
52. Sottnik J L, et al. (2013) Integrin alpha2beta 1 (alpha2beta1) promotes prostate cancer skeletal metastasis. Clin Exp Metastasis 30:569-578.
53. van der Bij G J, et al. (2008) Blocking alpha2 integrins on rat CC531s colon carcinoma cells prevents operation-induced augmentation of liver metastases outgrowth. Hepatology 47:532-543.
54. Tran T, et al. (2011) Loss of the alpha2beta1 integrin alters human papilloma virus-induced squamous carcinoma progression in vivo and in vitro. PLoS One 6:e26858.
55. Reticker-Flynn N E, et al. (2012) A combinatorial extracellular matrix platform identifies cell-extracellular matrix interactions that correlate with metastasis. Nat Commun 3:1122.
56. Vuoristo M, et al. (2007) Increased gene expression levels of collagen receptor integrins are associated with decreased survival parameters in patients with advanced melanoma. Melanoma Res 17:215-223.
57. Veit G, et al. (2011) Collagen XXIII, novel ligand for integrin alpha2beta1 in the epidermis. J Biol Chem 286:27804-27813.
58. Nieuwenhuis H K, Akkerman J W, Houdijk W P, & Sixma J J (1985) Human blood platelets showing no response to collagen fail to express surface glycoprotein Ia. Nature 318:470-472.
59. Blumbach K, et al. (2012) Dwarfism in mice lacking collagen-binding integrins alpha2beta1 and alpha11beta1 is caused by severely diminished IGF-1 levels. J Biol Chem 287:6431-6440.
60. Chen J, Diacovo T G, Grenache D G, Santoro S A, & Zutter M M (2002) The alpha(2) integrin subunit-deficient mouse: a multifaceted phenotype including defects of branching morphogenesis and hemostasis. Am J Pathol 161:337-344.
61. Holtkotter O, et al. (2002) Integrin alpha 2-deficient mice develop normally, are fertile, but display partially defective platelet interaction with collagen. J Biol Chem 277:10789-10794.
62. Halland N, Blum H, Buning C, Kohlmann M, & Lindenschmidt A (2014) Small Macrocycles As Highly Active Integrin α2β1 Antagonists. ACS Med Chem Lett 5:193-198.
63. Miller M W, et al. (2009) Small-molecule inhibitors of integrin alpha2beta1 that prevent pathological thrombus formation via an allosteric mechanism. Proc Natl Acad Sci USA 106:719-724.
64. Borza C M, et al. (2012) Inhibition of integrin alpha2beta1 ameliorates glomerular injury. J Am Soc Nephrol 23:1027-1038.
65. Grenache D G, et al. (2007) Wound healing in the alpha2beta1 integrin-deficient mouse: altered keratinocyte biology and dysregulated matrix metalloproteinase expression. J Invest Dermatol 127:455-466.
66. He L, et al. (2003) The contributions of the alpha 2 beta 1 integrin to vascular thrombosis in vivo. Blood 102:3652-3657.
67. Peters M A, et al. (2012) The loss of alpha2beta1 integrin suppresses joint inflammation and cartilage destruction in mouse models of rheumatoid arthritis. Arthritis Rheum 64:1359-1368.
68. San Antonio J D, et al. (2009) A key role for the integrin alpha2beta1 in experimental and developmental angiogenesis. Am J Pathol 175:1338-1347.
69. Schiro J A, et al. (1991) Integrin alpha 2 beta 1 (VLA-2) mediates reorganization and contraction of collagen matrices by human cells. Cell 67:403-410.
70. Zweers M C, et al. (2007) Integrin alpha2beta1 is required for regulation of murine wound angiogenesis but is dispensable for reepithelialization. J Invest Dermatol 127:467-478.
71. Quante M, et al. (2011) Bone marrow-derived myofibroblasts contribute to the mesenchymal stem cell niche and promote tumor growth. Cancer Cell 19:257-272.
72. Naba A, et al. (2014) Extracellular matrix signatures of human primary metastatic colon cancers and their metastases to liver. BMC cancer 14:518.
73. Grzesiak, J. J., Ho, J. C., Moossa, A. R., and Bouvet, M. (2007) The integrin-extracellular matrix axis in pancreatic cancer. Pancreas 35, 293-301.
74. Shield, K., Riley, C., Quinn, M. A., Rice, G. E., Ackland, M. L., and Ahmed, N. (2007) Alpha2beta1 integrin affects metastatic potential of ovarian carcinoma spheroids by supporting disaggregation and proteolysis. J Carcinog 6, 11.
75. Yoshimura, K., Meckel, K. F., Laird, L. S., Chia, C. Y., Park, J. J., Olino, K. L., Tsunedomi, R., Harada, T., Iizuka, N., Hazama, S., et al. (2009) Integrin alpha2 mediates selective metastasis to the liver. Cancer Res 69, 7320-7328.
76. Canon, J., Bryant, R., Roudier, M., Osgood, T., Jones, J., Miller, R., Coxon, A., Radinsky, R., and Dougall, W. C. (2010). Inhibition of RANKL increases the anti-tumor effect of the EGFR inhibitor panitumumab in a murine model of bone metastasis. Bone 46, 1613-1619.
77. Canon, J. R., Roudier, M., Bryant, R., Morony, S., Stolina, M., Kostenuik, P. J., and Dougall, W. C. (2008). Inhibition of RANKL blocks skeletal tumor progression and improves survival in a mouse model of breast cancer bone metastasis. Clin Exp Metastasis 25, 119-129.
78. Emsley, J., Knight, C. G., Farndale, R. W., Barnes, M. J., and Liddington, R. C. (2000). Structural basis of collagen recognition by integrin alpha2beta1. Cell 101, 47-56.
79. Grzesiak, J. J., Ho, J. C., Moossa, A. R., and Bouvet, M. (2007). The integrin-extracellular matrix axis in pancreatic cancer. Pancreas 35, 293-301.
80. Kirkland, S. C. (2009). Type I collagen inhibits differentiation and promotes a stem cell like phenotype in human colorectal carcinoma cells. Br J Cancer 101, 320-326.
81. Shield, K., Riley, C., Quinn, M. A., Rice, G. E., Ackland, M. L., and Ahmed, N. (2007). Alpha2beta1 integrin affects metastatic potential of ovarian carcinoma spheroids by supporting disaggregation and proteolysis. J Carcinog 6, 11.
82. Van Slambrouck, S., Jenkins, A. R., Romero, A. E., and Steelant, W. F. (2009). Reorganization of the integrin alpha2 subunit controls cell adhesion and cancer cell invasion in prostate cancer. Int J Oncol 34, 1717-1726.
83. Yoshimura, K., Meckel, K. F., Laird, L. S., Chia, C. Y., Park, J. J., Olino, K. L., Tsunedomi, R., Harada, T., Iizuka, N., Hazama, S., et al. (2009). Integrin alpha2 mediates selective metastasis to the liver. Cancer Res 69, 7320-7328.

Each of the references cited herein is incorporated by reference in its entirety or in relevant part, as would be apparent from the context of the citation.

Numerous modifications and variations of the disclosure are possible in view of the above teachings and are within the scope of the claims. The above-described embodiments are not intended to limit the claims in any way. The entire disclosures of all publications cited herein are hereby incorporated by reference.

What is claimed is:

1. A method of eliciting an antibody specifically binding a target comprising
(a) administering an effective amount of a three-dimensional hydrogel comprising a biomolecular target molecule; and
(b) obtaining an antibody that specifically binds to the target molecule.

2. The method according to claim 1 wherein the hydrogel comprises type I collagen, fibrin, or a mixture thereof.

3. The method according to claim 2 wherein the hydrogel comprises type I collagen.

4. The method according to claim 2 wherein the type I collagen, fibrin, or a mixture thereof is cross-linked.

5. The method according to claim 1 wherein the biomolecular target molecule is a cell-surface protein.

6. The method according to claim 5 wherein the cell-surface protein is on the surface of a diseased cell.

7. The method according to claim 6 wherein the diseased cell is a cancer cell, a fibrotic cell, an inflammatory cell, an immune cell or a cell participating in pathologic angiogenesis.

8. The method according to claim 6 wherein the diseased cell is a cancer cell or a fibrotic cell.

9. The method according to claim 1 wherein the biomolecular target molecule is α2 integrin, α-enolase, calnexin, CD44, filamin, vimentin, or fibrinogen.

10. The method according to claim 1 further comprising a subtractive immunization procedure comprising
(a) administering an effective amount of a hydrogel comprising a healthy cell that is a counterpart to the the cell associated with a disease, disorder or condition, to a host organism to elicit an antibody response; and
(b) delivering an immunosuppressive agent to the host organism.

11. The method according to claim 10 wherein the immunosuppressive agent is cyclophosphamide.

12. A method of producing an immunogen comprising
(a) obtaining a composition comprising a biomolecular target molecule;
(b) combining the composition comprising the biomolecular target molecule and a hydrogel-forming compound; and
(c) preparing a three-dimensional hydrogel comprising the composition comprising the biomolecular target molecule.

13. The method according to claim 12 wherein the hydrogel comprises type I collagen, fibrin, or a mixture thereof.

14. The method according to claim 13 wherein the type I collagen, fibrin, or a mixture thereof is cross-linked.

15. The method according to claim 12 wherein the biomolecular target molecule is a cell-surface protein.

16. The method according to claim 13 wherein the cell-surface protein is on the surface of a diseased cell.

17. The method according to claim 16 wherein the diseased cell is a cancer cell or a fibrotic cell.

18. The method according to claim 12 wherein the biomolecular target molecule is α2 integrin, α-enolase, calnexin, CD44, filamin, vimentin, or fibrinogen.

19. A method of identifying an anti-cancer antibody product functional in vivo comprising
(a) contacting a protein capable of cross-linking to form a hydrogel with a cancer cell to produce a hydrogel comprising a cancer cell;
(b) incubating the hydrogel comprising a cancer cell; and
(c) exposing the hydrogel comprising a cancer cell to an anti-cancer antibody product candidate under conditions suitable for antigen-antibody product binding,
wherein binding between the anti-cancer antibody product candidate and the hydrogel comprising a cancer cell identifies the anti-cancer antibody product candidate as an anti-cancer antibody product.

20. The method according to claim 19 wherein the cross-linked protein is a cross-linked matrix protein.

21. The method according to claim 20 wherein the matrix protein is type I collagen, elastin, or a mixture thereof.

22. The method according to claim 21 wherein the matrix protein is type I collagen.

23. The method according to claim 19 wherein the protein is modified to produce an aldimine derivative of the protein and the aldimine derivative of the protein produces the cross-linked protein.

24. The method according to claim 23 wherein lysyl oxidase catalyzes the modification of the protein to produce the aldimine derivative of the protein.

25. The method according to claim 19 wherein the hydrogel further comprises an α2 integrin holoprotein.

26. The method according to claim 25 wherein the α2 integrin holoprotein is α2 β1 integrin.

27. The method according to claim 19 wherein the hydrogel further comprises the α2 subunit of α2 β1 integrin.

28. The method according to claim 19 wherein the antibody product is a polyclonal antibody, a monoclonal antibody, an antibody fragment, a hybrid antibody, a chimeric antibody, a CDR-grafted antibody, a single chain antibody, a single chain variable fragment antibody, a Fab antibody fragment, a Fab′ antibody fragment, a F(ab′)2 antibody fragment, a linear antibody, a bi-body, a tri-body, a tetrabody, a diabody, a peptibody, a bispecific antibody, a bispecific T-cell engaging (BiTE) antibody, or a chimeric antibody receptor.

29. The method according to claim 28 wherein the antibody product is a humanized or human antibody product.

30. An antibody product produced by the method according to claim 19, wherein the antibody product is derived from the 4C3 monoclonal antibody.

31. The antibody product according to claim 28 wherein the antibody product is the 4C3 monoclonal antibody.

32. A seeded hydrogel comprising
(a) a cross-linked protein; and
(b) an integrin protein.

33. The hydrogel according to claim 32 wherein the cross-linked protein is a matrix protein.

34. The hydrogel according to claim 33 wherein the matrix protein is type I collagen, type III collagen, type IV collagen, fibrin, elastin, hyaluronic acid, laminin, or a mixture thereof.

35. The hydrogel according to claim 32 wherein the integrin protein is α2 β1 integrin.

36. The hydrogel according to claim 32 wherein the integrin protein is the α2 subunit of α2 β1 integrin.

37. The seeded hydrogel according to claim 32 further comprising a cell exhibiting a disease, disorder or condition.

38. The seeded hydrogel according to claim 37 wherein the cell is a cancer cell, a fibrotic cell, an inflammatory cell, an immune cell, an endothelial cell, a pericyte, a smooth muscle cell or a mesenchymal stem cell.
